(19) United States
(12) Patent Application Publication
(10) Pub. No.: US 2025/0149147 A1
(43) Pub. Date: May 8, 2025

(54) METHOD FOR DETERMINING THE GUT MICROBIOME STATUS

(71) Applicant: SOCIETE DES PRODUITS NESTLE S.A., Vevey (CH)

(72) Inventors: SHAILLAY KUMAR DOGRA, Epalinges (CH); FRANCIS FOATA, Belmont-sur-Lausanne (CH); JELENA BANJAC, Novi Sad (RS); NORBERT SPRENGER, Savigny (CH)

(21) Appl. No.: 18/833,062

(22) PCT Filed: Jan. 26, 2023
(86) PCT No.: PCT/EP2023/051932

§ 371 Date: Jul. 25, 2024

(30) Foreign Application Priority Data
Jan. 26, 2022 (EP) 22153503.2

(51) Int. Cl. G16H 20/60; G16B 40/20

(52) U.S. Cl. CPC G16H 20/60; G16B 40/20

(57) ABSTRACT

The present invention provides methods for determining the gut microbiome status of a subject given one or more CAZyme abundances provided from the subject's gut metagenomic data. The present invention also provides methods for maintaining or improving the gut microbiome status of a subject.

### FIELD OF THE INVENTION

The present invention relates to methods for determining the gut microbiome status of a subject. The present invention also relates to methods for maintaining or improving the gut microbiome status of a subject.

### BACKGROUND TO THE INVENTION

The microbiome can refer to both the composition of microorganisms in an ecosystem and their “theatre of activity”, which may include their structural elements (nucleic acids, proteins, lipids, polysaccharides), metabolites (signalling molecules, toxins, organic, and inorganic molecules), and molecules produced by coexisting hosts and structured by the surrounding environmental conditions (Berg, G., et al., 2020. Microbiome, 8(1), pp. 1-22).

The gut microbiome changes rapidly and dramatically in the first years of life (Yatsunenko, T., et al., 2012. Nature, 486(7402), pp. 222-227). For infants, certain factors such as birth mode, antibiotics usage and duration of exclusive breast-feeding impact the gut microbiome. Some other factors such as living location, siblings and furry pets can also influence the infant's gut microbiome (Dogra S. K., et al. Microorganisms 2021). Importantly, an infant's gut microbiome establishment has lasting consequences on their later health (Dogra S. K., et al. Gut Microbes. 2015; 6(5):321-5). Thus, it is important to promote age-appropriate gut microbiome maturation in infants.

Different approaches have been used to study microbiome maturation in early life. However, these approaches do not investigate the functional maturation of the microbiome ecosystem in the gut. Known approaches are usually bacteria composition based, indicating the changes in bacterial taxa, but not providing any functional information. It is challenging to use such approaches to provide personalised recommendations and nutritional interventions to promote age-appropriate gut microbiome maturation.

### SUMMARY OF THE INVENTION

The inventors have developed an approach for providing a trajectory of the early life microbiome (ELM) development using functional data. The inventors have shown that the abundance of carbohydrate-active enzymes (CAZymes) in gut metagenomic data can be used to generate an ELM trajectory. The inventors have identified the important CAZymes in a reference ELM trajectory and shown how these important CAZymes change over early life in the infant.

Further still, the inventors have described how CAZyme abundances can be adjusted to keep on or bring back a subject to the ELM trajectory. Personalised recommendations and dietary advice, such as modulating the amount of CAZyme-associated carbohydrates in the infant's diet, may be given to an infant's caretaker(s) to maintain or bring back the said infant on the ELM trajectory.

In one aspect, the present invention provides a method for providing a trained regression model for determining the gut microbiome status of a subject, wherein the method comprises: (a) providing gut metagenomic data from a population of healthy subjects; and (b) training a regression model on the gut metagenomic data, wherein the age of the healthy subjects at data collection is regressed on one or more carbohydrate-active enzyme (CAZyme) abundances provided from the gut metagenomic data.

The age of the healthy subjects at data collection may be regressed on a plurality of CAZyme abundances provided from the gut metagenomic data. In some embodiments, the age of the healthy subjects at data collection is regressed on 5 or more CAZyme abundances, 10 or more CAZyme abundances, 20 or more CAZyme abundances, 50 or more CAZyme abundances, or 100 or more CAZyme abundances. In some embodiments, the method further comprises determining the CAZyme abundances from the gut metagenomic data. In some embodiments, the CAZyme abundances are relative CAZyme abundances, suitably wherein the CAZyme abundances are relative to total bacterial genes.

The CAZymes may be any microbial CAZyme. The CAZymes may be selected from glycoside hydrolases (GH), glycosyltransferases (GT), polysaccharide lyases (PL), carbohydrate esterases (CE), and their associated carbohydrate binding modules (CBMs). Suitably, the CAZymes are classified by clans, families, and/or sub-families. Suitably, the CAZymes are classified by families. Suitably, the CAZymes are selected from one or more of: GH1, GH2, GH3, GH4, GH5, GH6, GH7, GH8, GH9, GH10, GH11, GH12, GH13, GH14, GH15, GH16, GH17, GH18, GH19, GH20, GH21, GH22, GH23, GH24, GH25, GH26, GH27, GH28, GH29, GH30, GH31, GH32, GH33, GH34, GH35, GH36, GH37, GH38, GH39, GH40, GH41, GH42, GH43, GH44, GH45, GH46, GH47, GH48, GH49, GH50, GH51, GH52, GH53, GH54, GH55, GH56, GH57, GH58, GH59, GH60, GH61, GH62, GH63, GH64, GH65, GH66, GH67, GH68, GH69, GH70, GH71, GH72, GH73, GH74, GH75, GH76, GH77, GH78, GH79, GH80, GH81, GH82, GH83, GH84, GH85, GH86, GH87, GH88, GH89, GH90, GH91, GH92, GH93, GH94, GH95, GH96, GH97, GH98, GH99, GH100, GH101, GH102, GH103, GH104, GH105, GH106, GH107, GH108, GH109, GH110, GH111, GH112, GH113, GH114, GH115, GH116, GH117, GH118, GH119, GH120, GH121, GH122, GH123, GH124, GH125, GH126, GH127, GH128, GH129, GH130, GH131, GH132, GH133, GH134, GH135, GH136, GH137, GH138, GH139, GH140, GH141, GH142, GH143, GH144, GH145, GH146, GH147, GH148, GH149, GH150, GH151, GH152, GH153, GH154, GH155, GH156, GH157, GH158, GH159, GH160, GH161, GH162, GH163, GH164, GH165, GH166, GH167, GH168, GH169, GH170, GH171, GH172, GT1, GT2, GT3, GT4, GT5, GT6, GT7, GT8, GT9, GT10, GT11, GT12, GT13, GT14, GT15, GT16, GT17, GT18, GT19, GT20, GT21, GT22, GT23, GT24, GT25, GT26, GT27, GT28, GT29, GT30, GT31, GT32, GT33, GT34, GT35, GT36, GT37, GT38, GT39, GT40, GT41, GT42, GT43, GT44, GT45, GT46, GT47, GT48, GT49, GT50, GT51, GT52, GT53, GT54, GT55, GT56, GT57, GT58, GT59, GT60, GT61, GT62, GT63, GT64, GT65, GT66, GT67, GT68, GT69, GT70, GT71, GT72, GT73, GT74, GT75, GT76, GT77, GT78, GT79, GT80, GT81, GT82, GT83, GT84, GT85, GT86, GT87, GT88, GT89, GT90, GT91, GT92, GT93, GT94, GT95, GT96, GT97, GT98, GT99, GT100, GT101, GT102, GT103, GT104, GT105, GT106, GT107, GT108, GT109, GT110, GT111, GT112, GT113, GT114, PL1, PL2, PL3, PL4, PL5, PL6, PL7, PL8, PL9, PL10, P11, PL12, PL13, PL14, PL15, PL16, PL17, PL18, PL19, PL20, PL21, PL22, PL23, PL24, PL25, PL26, PL27, PL28, PL29, PL30, PL31, PL32, PL33, PL34, PL35, PL36, PL37, PL38, PL39, PL40, PL41, PL42, CE1, CE2, CE3, CE4, CE5, CE6, CE7, CE8, CE9, CE10, CE11, CE12, CE13, CE14, CE15, CE16, CE17, CE18, CE19, CBM1, CBM2, CBM3, CBM4, CBM5, CBM6, CBM7, CBM8, CBM9, CBM10, CBM11, CBM12, CBM13, CBM14, CBM15, CBM16, CBM17, CBM18, CBM19, CBM20, CBM21, CBM22, CBM23, CBM24, CBM25, CBM26, CBM27, CBM28, CBM29, CBM30, CBM31, CBM32, CBM33, CBM34, CBM35, CBM36, CBM37, CBM38, CBM39, CBM40, CBM41, CBM42, CBM43, CBM44, CBM45, CBM46, CBM47, CBM48, CBM49, CBM50, CBM51, CBM52, CBM53, CBM54, CBM55, CBM56, CBM57, CBM58, CBM59, CBM60, CBM61, CBM62, CBM63, CBM64, CBM65, CBM66, CBM67, CBM68, CBM69, CBM70, CBM71, CBM72, CBM73, CBM74, CBM75, CBM76, CBM77, CBM78, CBM79, CBM80, CBM81, CBM82, CBM83, CBM84, CBM85, CBM86, CBM87, and CBM88.

The trained regression model may be an Early Life Microbiome (ELM) trajectory. The trained regression model may predict the age of a subject given their gut metagenomic data. Suitably, the trained regression model relates the age of a healthy subject to their microbiome age, microbiome maturation index, and/or microbiome maturation age.

The trained regression model may be for infancy and/or early childhood. In some embodiments, the trained regression model is for 0-5 years of age, 0-3 years of age, 0-2 years of age. In some embodiments, the trained regression model is for 0-24 months of age.

In some embodiments, the method further comprises obtaining the gut metagenomic data from the population of healthy subjects. Suitably, the gut metagenomic data is obtained or obtainable from fecal samples. Suitably, the gut metagenomic data is obtained or obtainable by next-generation sequencing.

The healthy subjects may be infants and/or children. In some embodiments, the healthy subjects are 0-5 years of age, or 0-3 years of age, or 0-2 years of age. In some embodiments, the healthy subjects are 0-24 months of age. Suitably, the population of healthy subjects comprises at least 20 healthy subjects, at least 40 healthy subjects, at least 60 healthy subjects, at least 80 healthy subjects, or at least 100 healthy subjects. Suitably, the gut metagenomic data from a population of healthy subjects comprises at least 50 samples, at least 100 samples, at least 200 samples, at least 300 samples, at least 400 samples, or at least 500 samples.

The regression model may be a tree-based regression model, such as a random forest regression model. In some embodiments, the age of the healthy subjects at sample collection is also regressed on one or more additional features provided from the gut metagenomic data.

In one aspect, the present invention provides a trained regression model obtained or obtainable by a method according to the present invention.

In one aspect, the present invention provides a trained regression model for determining the gut microbiome status of a subject given one or more CAZyme abundances provided from the subject's gut metagenomic data. Suitably, the trained regression model is obtained or is obtainable by a method according to the present invention.

In one aspect, the present invention provides a method for predicting the age of a subject, wherein the method comprises: (a) providing a trained regression model by a method according to the present invention, or a trained regression model according to the present invention; (b) providing gut metagenomic data from the subject; and (c) predicting the age of the subject given their gut metagenomic data and the trained regression model.

In one aspect, the present invention provides a method for determining the gut microbiome status of a subject, wherein the method comprises: (a) providing a trained regression model by a method according to the present invention, or a trained regression model according to the present invention; (b) providing gut metagenomic data from the subject; and (c) determining whether the subject is an outlier or not in the trained regression model; wherein the gut microbiome status of the subject is healthy if the subject is not an outlier in the trained regression model, and/or wherein the gut microbiome status of the subject is not healthy if the subject is an outlier in the trained regression model.

In some embodiments, the subject is an outlier based on the standard errors (SE), confidence intervals, prediction intervals, and/or standard deviations in the trained regression model. In some embodiments, the subject is an outlier if their gut metagenomic data is −2SE or less or 2SE or more from the trained regression line, if their gut metagenomic data falls outside the 95% confidence interval in the trained regression model, if their gut metagenomic data falls outside the 95% prediction interval in the trained regression model, and/or if they have a Z-score of −2 or less or 2 or more in the trained regression model. In some embodiments, the subject is an outlier if their gut metagenomic data falls outside the 95% prediction interval in the trained regression model.

In one aspect, the present invention provides a method for determining the gut microbiome status of a subject, wherein the method comprises: (a) providing a trained regression model by a method according to the present invention, or a trained regression model according to the present invention, wherein the trained regression model is an ELM trajectory; (b) providing gut metagenomic data from the subject; and (c) determining whether the subject is on or off the ELM trajectory; wherein the gut microbiome status of the subject is healthy if the subject is on the ELM trajectory, and/or wherein the gut microbiome status of the subject is not healthy if the subject is off the ELM trajectory. Suitably, the subject is on the ELM trajectory if the subject's gut metagenomic data does not differ significantly from the ELM trajectory and/or wherein the subject is off the ELM trajectory if the subject's gut metagenomic data differs significantly from the ELM trajectory.

In some embodiments, the subject is determined to be off the ELM trajectory based on the standard errors (SE), confidence intervals, prediction intervals, and/or standard deviations of the ELM trajectory. In some embodiments, the subject is determined to be off the ELM trajectory if their gut metagenomic data is −2SE or less or 2SE or more from the ELM trajectory, if their gut metagenomic data falls outside the 95% confidence interval of the ELM trajectory, if their gut metagenomic data falls outside the 95% prediction interval of the ELM trajectory, and/or if they have a Z-score of −2 or less or 2 or more. In some embodiments, the subject is determined to be off the ELM trajectory if their gut metagenomic data falls outside the 95% prediction interval of the ELM trajectory.

In one aspect, the present invention provides a method for determining the gut microbiome status of a subject, wherein the method comprises predicting the age of the subject by a method according to the present invention, and wherein the gut microbiome status of the subject is healthy if the predicted age of the subject does not differ significantly from the actual age of the subject and/or wherein the gut microbiome status of the subject is not healthy if the predicted age of the subject differs significantly from the actual age of the subject.

In some embodiments, the predicted age of the subject differs significantly from the actual age of the subject if the predicted age of the subject if it differs by about 0.5 years or more, by about 0.6 years or more, by about 0.7 years or more, by about 0.8 years or more, by about 0.9 years or more, or by about 1 year or more.

Suitably, in a method for predicting the age of the subject or determining the gut microbiome status according to the present invention, the subject's gut metagenomic data is obtained or obtainable from fecal samples and/or by next-generation sequencing. In some embodiments, the subject's gut metagenomic may provide 5 or more CAZyme abundances, 10 or more CAZyme abundances, 20 or more CAZyme abundances, 50 or more CAZyme abundances, or 100 or more CAZyme abundances.

In one aspect, the present invention provides a method for maintaining or improving the gut microbiome status of a subject, wherein the method comprises: (a) determining the gut microbiome status of the subject by a method according to the present invention; and (b) adjusting the diet, nutrient intake, and/or lifestyle of the subject to maintain or improve the subject's gut microbiome status. After adjusting the diet, nutrient intake, and/or lifestyle of the subject the gut microbiome status of the subject may be healthy.

The adjusted diet, nutrient intake, and/or lifestyle of the subject may increase the abundance of favourable CAZymes and/or decrease the abundance of unfavourable CAZymes.

In one aspect, the present invention provides a method for determining a subject's diet and/or nutrient intake, wherein the method comprises: (a) determining the gut microbiome status of the subject by a method according to the present invention; and (b) determining the diet and/or nutrient intake required to maintain or improve the gut microbiome status of the subject.

Suitably, the subject is administered food and/or supplements to increase the abundance of favourable CAZymes and/or to decrease the abundance of unfavourable CAZymes. The food and/or supplements may comprise one or more carbohydrates. Suitably, the carbohydrates may be selected from one or more of: 4-methylumbelliferyl 6-azido-6-deoxy-beta-D-galactoside, N-acetyl-D-galactosamine, N-acetyl-D-glucosamine, N-acetylglucosamine, N-glycan, O-antigen, O-glycan, acarbose, acetylated glucuronoxylan, agar, aldouronate, alginate, alpha-galactoside, alpha-glucan, alpha-mannan, alpha-mannoside, arabinan, arabino-xylooligosaccharide, arabinofuranooligosaccharide, arabinofuranose, arabinogalactan, arabinose, arabinoxylan, beta-galactooligosaccharide, beta-galactoside, beta-glucan, beta-glucoside, beta-mannan, capsule polysaccharide, carboxymethylcellulose, carrageenan, cellobiose, cellodextrin, cellooligosaccharide, cellotriose, cellulose, chitin, chitobiose, chitooligosaccharide, chondroitin disaccharide, chondroitin sulfate, chondrotin sulfate, cyclomaltodextrin, d-galactosamine, dextran, emulsan, exopolysaccharide, fructan, fructooligosaccharide, fucose, fucosyllactose, galactan, galactomannan, galactooligosaccharide, galactose, galacturonic acid, gentiobiose, glucan, glucomannan, glucosamine, glucose, glycogen, glycosaminoglycan, hemicellulose, heparosan, homogalacturonan, host glycan, human milk oligosaccharide, inulin, isomaltose, isomaltotriose, kestose, lacto-n-triose, lactose, laminaribiose, laminarin, levan, lichenan, lignocellulose, lipopolysaccharide, maltodextrin, maltooligosaccharide, maltose, maltotriose, mannan, mannooligosaccharide, mannose, melezitose, melibiose, methylglucuronoarabinoxylan, mucin, oligogalacturonide, outer core capsule polysaccharide, panose, pectin, plant polysaccharide, polygalacturonic acid, polysialic acid, porphyran, pullulan, raffinose-oligosaccharide, raffinose, rhamnogalacturonan, rhamnose, ribose, sialic acid, sialoglycoconjugate, sophorose, sorbitol, stachyose, starch, sucrose, trehalose, ulvan, unsaturated hyaluronate disaccharide, xanthan, xylan, xylobiose, xylodextrin, xyloglucan, xylooligosaccharide, xylose, and xylotriose.

In one aspect, the present invention provides a data processing system comprising means for carrying out the method according to the present invention.

In one aspect, the present invention provides a data processing system comprising a processor configured to perform the method according to the present invention.

In one aspect, the present invention provides a computer program comprising instructions which, when the program is executed by a computer, cause the computer to carry out the method according to the present invention.

In one aspect, the present invention provides a computer program comprising instructions which, when the program is executed by a computer, cause the computer to determine a trained regression model for determining the gut microbiome status of a subject from a population of healthy subjects, given the age of the healthy subjects at data collection and one or more CAZyme abundances provided from the healthy subject's gut metagenomic data.

In one aspect, the present invention provides a computer program comprising instructions which, when the program is executed by a computer, cause the computer to predict the age of a subject or determine the gut microbiome status of a subject, given one or more CAZyme abundances provided from the subject's gut metagenomic data.

In one aspect, the present invention provides a computer program comprising instructions which, when the program is executed by a computer, cause the computer to predict the age of a subject or determine the gut microbiome status of a subject, given a regression model trained on the gut metagenomic data from a population of healthy subjects and the subject's gut metagenomic data, wherein the regression model was trained by regressing the age of the healthy subjects at data collection on one or more CAZyme abundances provided from the healthy subject's gut metagenomic data. In some embodiments, the trained regression model is a trained regression model according to the present invention.

In one aspect, the present invention provides a computer-readable medium comprising instructions which, when executed by a computer, cause the computer to carry out the method according to the present invention.

In one aspect, the present invention provides a computer-readable medium comprising instructions which, when executed by a computer, cause the computer to determine a trained regression model for determining the gut microbiome status of a subject from a population of healthy subjects, given the age of the healthy subjects at data collection and one or more CAZyme abundances provided from the healthy subject's gut metagenomic data.

In one aspect, the present invention provides a computer-readable medium comprising instructions which, when the program is executed by a computer, cause the computer to predict the age of a subject or determine the gut microbiome status of a subject, given one or more CAZyme abundances provided from the subject's gut metagenomic data.

In one aspect, the present invention provides a computer-readable medium comprising instructions which, when executed by a computer, cause the computer to predict the age of a subject or determine the gut microbiome status of a subject, given a regression model trained on the gut metagenomic data from a population of healthy subjects and the subject's gut metagenomic data, wherein the regression model was trained by regressing the age of the healthy subjects at data collection on one or more CAZyme abundances provided from the healthy subject's gut metagenomic data. In some embodiments, the trained regression model is a trained regression model according to the present invention.

In one aspect, the present invention provides a computer-readable data carrier having stored thereon a computer program according to the present invention.

In one aspect, the present invention provides a data carrier signal carrying a computer program according to the present invention.

In one aspect, the present invention provides use of one or more CAZyme abundances provided from a subject's gut metagenomic data to predict the age of the subject or to determine the gut microbiome status of the subject.

In one aspect, the present invention provides use of one or more CAZyme abundances provided from gut metagenomic data from a population of healthy subjects to train a regression model.

In one aspect, the present invention provides use of a trained regression model according to the present invention to predict the age of a subject or to determine the gut microbiome status of a subject.

Suitably, the subject of interest is an infant or a child. Suitably, the subject of interest is 0-5 years of age, or 0-3 years of age, or 0-2 years of age. Suitably, the subject of interest is 0-24 months of age.

### DESCRIPTION OF DRAWINGS

FIG. 1—Early Life Microbiome (ELM) trajectory derived using CAZyme abundances

An Early Life Microbiome (ELM) trajectory obtained using CAZyme abundances provided from the gut metagenomic data of Reference subjects is shown.

FIG. 2—ELM trajectory showing the key CAZyme abundances per time window

The same ELM trajectory shown in FIG. 1, wherein now the key CAZyme abundances per different time windows are shown.

FIG. 3—bringing an outlier back on to the CAZymes based ELM trajectory

An outlier is brought back on to the ELM trajectory by adjusting the abundance of CAZymes in the subject's metagenomic data.

FIG. 4—Reference vs. non-Reference CAZymes based ELM trajectory

Clear differences are seen in CAZymes based ELM trajectory for Reference subjects vs. non-Reference subjects.

### DETAILED DESCRIPTION

Various preferred features and embodiments of the present invention will now be described by way of non-limiting examples. The skilled person will understand that they can combine all features of the invention disclosed herein without departing from the scope of the invention as disclosed.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

Numeric ranges are inclusive of the numbers defining the range.

The publications discussed herein are provided solely for their disclosure prior to the filing date of the present application. Nothing herein is to be construed as an admission that such publications constitute prior art to the claims appended hereto.

The methods and systems disclosed herein can be used by doctors, health-care professionals, lab technicians, infant care providers and so on.

### Method for Providing a Trained Regression Model

The present invention provides a method for providing a trained regression model for determining the gut microbiome status of a subject. The present invention also provides a trained regression model obtained or obtainable by such a method.

### Microbiota and Microbiome

The “gut microbiota” may refer to the composition of microorganisms (including bacteria, archaea and fungi) that live in the digestive tract.

The term “gut microbiome” may encompass both the “gut microbiota” and their “theatre of activity”, which may include their structural elements (nucleic acids, proteins, lipids, polysaccharides), metabolites (signalling molecules, toxins, organic, and inorganic molecules), and molecules produced by coexisting hosts and structured by the surrounding environmental conditions (see e.g. Berg, G., et al., 2020. Microbiome, 8(1), pp. 1-22).

In the present invention, the term “gut microbiome” may therefore be used interchangeably with the term “gut microbiota”.

A subject's “gut microbiome status” may refer to the state, condition, or development of a subject's gut microbiome at a particular time. The term “gut microbiome status” may refer to a subject's microbiome age, microbiome maturation index, and/or Early Life Microbiome (ELM) trajectory.

A subject's “microbiome age” can refer to the predicted age of a subject based on their gut microbiome data. For example, the actual/chronological age of subject may be predicted from gut metagenomic data using machine learning based artificial intelligence approaches. A subject's “microbiome maturation index” or “microbiome maturation age” may be obtained as above.

A subject's “ELM trajectory” may refer to a fitted curve which is obtained to describe the relation of “microbiome age” or “microbiome maturation index” with actual age. The curve may be fitted by methods such as LOESS or smooth splines using another cohort or subset of data (for external validation purposes).

### Carbohydrate-Active Enzymes (CAZymes)

The present invention uses one or more CAZyme abundances to train the regression model and to determine the gut microbiome status of a subject.

Carbohydrate-active enzymes (CAZymes) may refer to enzymes involved in the synthesis, metabolism, and transport of carbohydrates. CAZymes may include glycoside hydrolases (GHs), glycosyltransferases (GTs), polysaccharide lyases (PLs), carbohydrate esterases (CEs) and carbohydrate binding modules (CBMs). The CAZy database (http://www.cazy.org) is a dedicated CAZyme classification system which is widely used by the scientific community. Close to 300 families of catalytic and ancillary modules are presented in CAZy and correspond to over 100,000 non-redundant entries.

Suitably, the CAZymes are microbial CAZymes. The CAZymes may be classified according to any suitable classification system, see e.g. Lombard, V., et al., 2014. Nucleic acids research, 42(D1), pp. D490-D495. The CAZymes may be classified by the same classification system(s) or by one or more different classification systems. Suitably, the CAZymes are classified by clans, families, and/or sub-families. Suitably, the CAZymes are classified by families.

Suitably, the CAZymes comprise, consist essentially of, or consist of one or more (e.g. 5 or more, 10 or more, 20 or more, 50 or more, or 100 or more) of: GH1, GH2, GH3, GH4, GH5, GH6, GH7, GH8, GH9, GH10, GH11, GH12, GH13, GH14, GH15, GH16, GH17, GH18, GH19, GH20, GH21, GH22, GH23, GH24, GH25, GH26, GH27, GH28, GH29, GH30, GH31, GH32, GH33, GH34, GH35, GH36, GH37, GH38, GH39, GH40, GH41, GH42, GH43, GH44, GH45, GH46, GH47, GH48, GH49, GH50, GH51, GH52, GH53, GH54, GH55, GH56, GH57, GH58, GH59, GH60, GH61, GH62, GH63, GH64, GH65, GH66, GH67, GH68, GH69, GH70, GH71, GH72, GH73, GH74, GH75, GH76, GH77, GH78, GH79, GH80, GH81, GH82, GH83, GH84, GH85, GH86, GH87, GH88, GH89, GH90, GH91, GH92, GH93, GH94, GH95, GH96, GH97, GH98, GH99, GH100, GH101, GH102, GH103, GH104, GH105, GH106, GH107, GH108, GH109, GH110, GH111, GH112, GH113, GH114, GH115, GH116, GH117, GH118, GH119, GH120, GH121, GH122, GH123, GH124, GH125, GH126, GH127, GH128, GH129, GH130, GH131, GH132, GH133, GH134, GH135, GH136, GH137, GH138, GH139, GH140, GH141, GH142, GH143, GH144, GH145, GH146, GH147, GH148, GH149, GH150, GH151, GH152, GH153, GH154, GH155, GH156, GH157, GH158, GH159, GH160, GH161, GH162, GH163, GH164, GH165, GH166, GH167, GH168, GH169, GH170, GH171, GH172, GT1, GT2, GT3, GT4, GT5, GT6, GT7, GT8, GT9, GT10, GT11, GT12, GT13, GT14, GT15, GT16, GT17, GT18, GT19, GT20, GT21, GT22, GT23, GT24, GT25, GT26, GT27, GT28, GT29, GT30, GT31, GT32, GT33, GT34, GT35, GT36, GT37, GT38, GT39, GT40, GT41, GT42, GT43, GT44, GT45, GT46, GT47, GT48, GT49, GT50, GT51, GT52, GT53, GT54, GT55, GT56, GT57, GT58, GT59, GT60, GT61, GT62, GT63, GT64, GT65, GT66, GT67, GT68, GT69, GT70, GT71, GT72, GT73, GT74, GT75, GT76, GT77, GT78, GT79, GT80, GT81, GT82, GT83, GT84, GT85, GT86, GT87, GT88, GT89, GT90, GT91, GT92, GT93, GT94, GT95, GT96, GT97, GT98, GT99, GT100, GT101, GT102, GT103, GT104, GT105, GT106, GT107, GT108, GT109, GT110, GT111, GT112, GT113, GT114, PL1, PL2, PL3, PL4, PL5, PL6, PL7, PL8, PL9, PL10, PL11, PL12, PL13, PL14, PL15, PL16, PL17, PL18, PL19, PL20, PL21, PL22, PL23, PL24, PL25, PL26, PL27, PL28, PL29, PL30, PL31, PL32, PL33, PL34, PL35, PL36, PL37, PL38, PL39, PL40, PL41, PL42, CE1, CE2, CE3, CE4, CE5, CE6, CE7, CE8, CE9, CE10, CE11, CE12, CE13, CE14, CE15, CE16, CE17, CE18, CE19, CBM1, CBM2, CBM3, CBM4, CBM5, CBM6, CBM7, CBM8, CBM9, CBM10, CBM11, CBM12, CBM13, CBM14, CBM15, CBM16, CBM17, CBM18, CBM19, CBM20, CBM21, CBM22, CBM23, CBM24, CBM25, CBM26, CBM27, CBM28, CBM29, CBM30, CBM31, CBM32, CBM33, CBM34, CBM35, CBM36, CBM37, CBM38, CBM39, CBM40, CBM41, CBM42, CBM43, CBM44, CBM45, CBM46, CBM47, CBM48, CBM49, CBM50, CBM51, CBM52, CBM53, CBM54, CBM55, CBM56, CBM57, CBM58, CBM59, CBM60, CBM61, CBM62, CBM63, CBM64, CBM65, CBM66, CBM67, CBM68, CBM69, CBM70, CBM71, CBM72, CBM73, CBM74, CBM75, CBM76, CBM77, CBM78, CBM79, CBM80, CBM81, CBM82, CBM83, CBM84, CBM85, CBM86, CBM87, and CBM88.

Suitably, the CAZymes comprise, consist essentially of, or consist of: GH1, GH2, GH3, GH4, GH5, GH6, GH7, GH8, GH9, GH10, GH11, GH12, GH13, GH14, GH15, GH16, GH17, GH18, GH19, GH20, GH21, GH22, GH23, GH24, GH25, GH26, GH27, GH28, GH29, GH30, GH31, GH32, GH33, GH34, GH35, GH36, GH37, GH38, GH39, GH40, GH41, GH42, GH43, GH44, GH45, GH46, GH47, GH48, GH49, GH50, GH51, GH52, GH53, GH54, GH55, GH56, GH57, GH58, GH59, GH60, GH61, GH62, GH63, GH64, GH65, GH66, GH67, GH68, GH69, GH70, GH71, GH72, GH73, GH74, GH75, GH76, GH77, GH78, GH79, GH80, GH81, GH82, GH83, GH84, GH85, GH86, GH87, GH88, GH89, GH90, GH91, GH92, GH93, GH94, GH95, GH96, GH97, GH98, GH99, GH100, GH101, GH102, GH103, GH104, GH105, GH106, GH107, GH108, GH109, GH110, GH111, GH112, GH113, GH114, GH115, GH116, GH117, GH118, GH119, GH120, GH121, GH122, GH123, GH124, GH125, GH126, GH127, GH128, GH129, GH130, GH131, GH132, GH133, GH134, GH135, GH136, GH137, GH138, GH139, GH140, GH141, GH142, GH143, GH144, GH145, GH146, GH147, GH148, GH149, GH150, GH151, GH152, GH153, GH154, GH155, GH156, GH157, GH158, GH159, GH160, GH161, GH162, GH163, GH164, GH165, GH166, GH167, GH168, GH169, GH170, GH171, GH172, GT1, GT2, GT3, GT4, GT5, GT6, GT7, GT8, GT9, GT10, GT11, GT12, GT13, GT14, GT15, GT16, GT17, GT18, GT19, GT20, GT21, GT22, GT23, GT24, GT25, GT26, GT27, GT28, GT29, GT30, GT31, GT32, GT33, GT34, GT35, GT36, GT37, GT38, GT39, GT40, GT41, GT42, GT43, GT44, GT45, GT46, GT47, GT48, GT49, GT50, GT51, GT52, GT53, GT54, GT55, GT56, GT57, GT58, GT59, GT60, GT61, GT62, GT63, GT64, GT65, GT66, GT67, GT68, GT69, GT70, GT71, GT72, GT73, GT74, GT75, GT76, GT77, GT78, GT79, GT80, GT81, GT82, GT83, GT84, GT85, GT86, GT87, GT88, GT89, GT90, GT91, GT92, GT93, GT94, GT95, GT96, GT97, GT98, GT99, GT100, GT101, GT102, GT103, GT104, GT105, GT106, GT107, GT108, GT109, GT110, GT111, GT112, GT113, GT114, PL1, PL2, PL3, PL4, PL5, PL6, PL7, PL8, PL9, PL10, P11, PL12, PL13, PL14, PL15, PL16, PL17, PL18, PL19, PL20, PL21, PL22, PL23, PL24, PL25, PL26, PL27, PL28, PL29, PL30, PL31, PL32, PL33, PL34, PL35, PL36, PL37, PL38, PL39, PL40, PL41, PL42, CE1, CE2, CE3, CE4, CE5, CE6, CE7, CE8, CE9, CE10, CE11, CE12, CE13, CE14, CE15, CE16, CE17, CE18, CE19, CBM1, CBM2, CBM3, CBM4, CBM5, CBM6, CBM7, CBM8, CBM9, CBM10, CBM11, CBM12, CBM13, CBM14, CBM15, CBM16, CBM17, CBM18, CBM19, CBM20, CBM21, CBM22, CBM23, CBM24, CBM25, CBM26, CBM27, CBM28, CBM29, CBM30, CBM31, CBM32, CBM33, CBM34, CBM35, CBM36, CBM37, CBM38, CBM39, CBM40, CBM41, CBM42, CBM43, CBM44, CBM45, CBM46, CBM47, CBM48, CBM49, CBM50, CBM51, CBM52, CBM53, CBM54, CBM55, CBM56, CBM57, CBM58, CBM59, CBM60, CBM61, CBM62, CBM63, CBM64, CBM65, CBM66, CBM67, CBM68, CBM69, CBM70, CBM71, CBM72, CBM73, CBM74, CBM75, CBM76, CBM77, CBM78, CBM79, CBM80, CBM81, CBM82, CBM83, CBM84, CBM85, CBM86, CBM87, and CBM88.

As used herein, a “CAZyme abundance” may refer to the abundance of a CAZyme gene in metagenomic data. The abundance may be a relative abundance and/or absolute abundance. Suitably, abundance is a relative abundance, for example, the abundances may be calculated relative to total bacterial genes (see e.g. Kaur, K., et al., 2020. PloS one, 15(4), p. e0231197) or relative to total CAZyme abundance. Suitably, the abundances are calculated relative to total bacterial genes.

Gut Metagenomic Data from a Population of Healthy Subjects

The method for providing a trained regression model may comprise providing gut metagenomic data from a population of healthy subjects. Suitably, the method further comprises obtaining the gut metagenomic data from the population of healthy subjects.

### Gut Metagenomic Data

A subject's “gut metagenomic data” or “gut metagenome data” may refer to all the genetic content of the subject's gut, including all the genomes and genes from the gut microbiota (see e.g. Berg, G., et al., 2020. Microbiome, 8(1), pp. 1-22; Pasolli, E., et al., 2019. Cell, 176(3), pp. 649-662; and Qin, J., et al., 2010. Nature, 464(7285), pp. 59-65).

The gut metagenomic data may provide one or more CAZyme abundances. Suitably, the gut metagenomic data may provide 2 or more (e.g. 2, 3, 4, 5, 6, 7, 8, 9, 10) CAZyme abundances, 3 or more CAZyme abundances, 4 or CAZyme abundances, 5 or more CAZyme abundances, 6 or more CAZyme abundances, 7 or more CAZyme abundances, 8 or more CAZyme abundances, 9 or more CAZyme abundances, 10 or more CAZyme abundances, 20 or more CAZyme abundances, 30 or more CAZyme abundances, 50 or more CAZyme abundances, 50 or more CAZyme abundances, or 100 or more CAZyme abundances. Suitably, the gut metagenomic data may provide 500 or fewer CAZyme abundances, 400 or fewer CAZyme abundances, 200 or fewer CAZyme abundances, or 100 or fewer CAZyme abundances. Suitably, the gut metagenomic data may provide from 2 to 500 CAZyme abundances, from 5 to 200 CAZyme abundances, or from 10 to 100 CAZyme abundances.

The gut metagenomic data may be obtained or obtainable by any suitable sampling method. For example, gut metagenomic data may be obtained or obtainable by any method described in Tang, Q., et al., 2020. Frontiers in cellular and infection microbiology, 10, p. 151. The gut metagenomic data may be obtained from or obtainable from fecal samples, endoscopy samples (e.g. biopsy samples, luminal brush samples, laser capture microdissection samples), aspirated intestinal fluid samples, surgery samples, or by in vivo models or intelligent capsule.

Suitably, the gut metagenomic data may be obtained from or obtainable from fecal samples. Fecal samples are naturally collected, non-invasive and can be sampled repeatedly. Fecal materials instantly frozen at −80° C. that can maintain microbial integrity without preservatives have been widely regarded as the gold standard for gut metagenomics, but other storage methods with or without preservatives can also be utilised to achieve metagenomic data similar to those of fresh samples.

The gut metagenomic data may be obtained by or obtainable from the samples by any suitable method. For example, the gut metagenomic data may be obtained by or obtainable from the samples by sequencing methods (e.g. next-generation sequencing (NGS) methods). NGS enables the profiling of the genomic DNA of all the microorganisms present in a sample. NGS methods can include shotgun sequencing approaches, e.g. as described in Poussin, C., et al., 2018. Drug discovery today, 23(9), pp. 1644-1657.

### Population of Healthy Subjects

The “population of healthy subjects” may refer to a population of subjects with no known underlying health conditions. In some embodiments, the population of subjects have no underlying health conditions. The healthy subjects may be human subjects. The subjects may be male and/or female.

The healthy subjects may be of any age. Suitably, the healthy subjects may be infants, toddlers and/or children. The term “infant” may refer to a subject aged from 0 years to 1 year, or from 0 months to less than 1 year. The term “toddler” may refer to a subject aged from 1 year to 3 years, or from 1 year to less than 3 years. The term “child” may refer to a subject aged under 18 years. The healthy subjects may be infants, toddler and/or young children. The term “young child” may refer to a subject aged from 3 years to 5 years, or from 3 years to less than 5 years.

Suitably, the healthy subjects are 5 years of age or less, 4 years of age or less, 3 years of age or less, 2 years of age or less, 1 year of age or less, or 0.5 years of age or less. Suitably, the healthy subjects are 60 months of age or less, 48 months of age or less, 36 months of age or less, 24 months of age or less, 12 months of age or less, or 6 months of age or less.

Suitably, the healthy subjects are 0 years of age or more, 0.5 years of age or more, or 1 year of age or more. Suitably, the healthy subjects are 0 months of age or more, 6 months of age or more, or 12 months of age or more.

Suitably, the healthy subjects are from 0 years to 5 years of age, from 0 years to 4 years of age, from 0 years to 3 years of age, from 0 years to 2 years of age, or from 0 years to 1 year of age. In some embodiments, the healthy subjects are from 0 years to 2 years of age. Suitably, the healthy subjects are from 0 months to 60 months of age, from 0 months to 48 months of age, from 0 months to 36 months of age, from 0 months to 24 months of age, or from 0 months to 12 months of age. In some embodiments, the healthy subjects are from 0 months to 24 months of age.

Suitably, the healthy subjects are from 0 years to 1 year of age, 0.5 years to 1 year of age, or 1 year to 2 years of age. Suitably, the healthy subjects are from 0 months to 12 months of age, 6 months to 12 months of age, or 12 months to 24 months of age.

The population of healthy subjects may comprise any number of subjects suitable for training a regression model. The population of healthy subjects may comprise at least 10 subjects, at least 20 subjects, at least 30 subjects, at least 40 subjects, at least 50 subjects, at least 60 healthy subjects, at least 80 healthy subjects, or at least 100 healthy subjects. Suitably, the population of healthy subjects may comprise 500 subjects or less, 100 subjects or less, or 50 subjects or less. Suitably, the population of healthy subjects may comprise from 10 to 500 subjects.

The gut metagenomic data from a population of healthy subjects may comprise any number of samples suitable for training a regression model. Suitably, the gut metagenomic data comprises at least 50 samples, at least 100 samples, at least 200 samples, at least 300 samples, at least 400 samples, at least 500 samples, or at least 1000 samples.

The gut metagenomic data from a population of healthy subjects may comprise any number of samples from any number of subjects which is suitable for training a regression model. Suitably, the gut metagenomic data from a population of healthy subjects may comprise at least 50 samples from at least 10 subjects.

### Regression Analysis

The present invention may use regression analysis to relate the age of a population of healthy subjects at metagenomic data collection to one or more CAZyme abundances provided from their metagenomic data.

Regression analysis is a set of statistical processes for estimating the relationships between a dependent variable (e.g. the age of the population of healthy subjects at data collection) and one or more independent variables (e.g. one or more CAZyme abundance from the gut metagenomic data). The regression analysis may be used to provide a trained (or fitted) regression model.

The regression analysis may be performed using be any suitable regression model. Suitable regression models will be well known to the skilled person. Exemplary regression models include decision tree regression, linear regression, polynomial regression, quantile regression, ridge regression, lasso regression, elastic net regression, and support vector regression.

Suitably, the regression analysis is performed using machine learning methods. Exemplary machine learning methods include tree-based regression models (e.g. a random forest regression models), recursive partitioning, regularized and shrinkage methods, boosting and gradient descent, and Bayesian methods.

Suitably, the regression model is a tree-based regression model (e.g. a random forest regression model). In some embodiments, the regression model is a random forest regression model.

The regression analysis may be performed by training a regression model on the gut metagenomic data. For example, regression analysis may be performed by training a regression model using the age of the healthy subjects at data collection and one or more CAZyme abundances provided from the gut metagenomic data.

As used herein, “training” of “fitting” a regression model may mean determining a function which most closely fits the data according to a suitable statistical criteria. For example, the method of ordinary least squares may be used to compute the function that minimizes the sum of squared differences between the true data and that function.

### Other Additional Features

The present invention may use one or more additional features (in addition to the one or more CAZyme abundances) to determine the gut microbiome status of a subject.

The term “features” includes responses obtained from the metagenomic data, other microbiome data, and/or the general metadata. The additional features may include, for example, the abundance of one or more microbial taxa.

For example, the method for providing a trained regression model may comprise training a regression model on gut metagenomic data from a population of healthy subjects, wherein the age of the healthy subjects at data collection is regressed on one or more CAZyme abundances provided from the gut metagenomic data and one or more additional features.

### Trained Regression Model

The present invention provides a trained regression model for determining the gut microbiome status of a subject given one or more CAZyme abundances provided from the subject's gut metagenomic data. The trained regression model may be obtained or obtainable by any method described herein.

The “trained regression model” (also known as a “fitted regression model”) may relate the age of a healthy subject to their metagenomic data. The trained regression model may provide a “trained regression function” or “trained regression line”, relating the age of a healthy subject to the one or more CAZyme abundances described herein, and other statistics such as the standard errors of the regression, confidence intervals, prediction intervals, and/or standard deviations of the regression.

The trained regression model may predict the age of a subject given their gut metagenomic data. For example, the trained regression model may predict the age of a subject given one or more CAZyme abundances described herein. The prediction may be based on assuming the subject is healthy.

The trained regression model may relate the age of a healthy subject to their microbiome age and/or microbiome maturation index. The trained regression model may be an Early Life Microbiome (ELM) trajectory.

The trained regression model may be for any age. For example, the trained regression model may be for infancy, toddlerhood and/or childhood. The term “infancy” may refer to from 0 years to 1 year of age, or from 0 months to less than 1 year of age. The term “toddlerhood” may refer to from 1 year to 3 years of age, or from 1 year to less than 3 years of age. The term “childhood” may refer to up to 18 years of age. The trained regression model may be for infancy, toddlerhood and/or early childhood. The term “early childhood” may refer to 3 years to 5 years of age, or from 3 years to less than 5 years of age.

Suitably, the trained regression model is for 5 years of age or less, 4 years of age or less, 3 years of age or less, 2 years of age or less, 1 year of age or less, or 0.5 years of age or less. Suitably, the trained regression model is for 60 months of age or less, 48 months of age or less, 36 months of age or less, 24 months of age or less, 12 months of age or less, or 6 months of age or less.

Suitably, the trained regression model is for 0 years of age or more, 0.5 years of age or more, or 1 year of age or more. Suitably, the trained regression model is for 0 months of age or more, 6 months of age or more, or 12 months of age or more.

Suitably, the trained regression model is for from 0 years to 5 years of age, from 0 years to 4 years of age, from 0 years to 3 years of age, from 0 years to 2 years of age, or from 0 years to 1 year of age. In some embodiments, the trained regression model is for from 0 years to 2 years of age. Suitably, the trained regression model is for from 0 months to 60 months of age, from 0 months to 48 months of age, from 0 months to 36 months of age, from 0 months to 24 months of age, or from 0 months to 12 months of age. In some embodiments, the trained regression model is for from 0 months to 24 months of age.

Suitably, the trained regression model is for from 0 years to 1 year of age, 0.5 years to 1 year of age, or 1 year to 2 years of age. Suitably, the trained regression model is for from 0 months to 12 months of age, 6 months to 12 months of age, or 12 months to 24 months of age.

The trained regression model will depend on the population of healthy subjects chosen. The trained regression model may vary, for example, depending on the age, diet, medication, birth mode, duration of exclusive breast-feeding, living location, ethnicity, siblings, and pets of the healthy population.

### Method for Determining the Gut Microbiome Status of a Subject

The present invention provides a method for determining the gut microbiome status of a subject of interest given their gut metagenomic data. The method may use any trained regression model described herein.

The present invention also provides use of one or more CAZymes abundances provided from a subject's gut metagenomic data to determine the gut microbiome status of the subject. The present invention also provides use of a trained regression model according to the present invention to determine the gut microbiome status of a subject.

### Subject of Interest

The subject of interest may be any suitable subject. For example, the subject of interest may be a subject with the same characteristics as the healthy population of subjects on which the regression model was trained (except wherein the subject may or may not be healthy). For example, the subject of interest may have an age which falls within the range of ages of the healthy population at data collection. The subject of interest may be human. The subject of interest may be male or female.

The subject of interest may be any age. For example, the subject of interest may be an infant, a toddler or a child. The subject of interest may be an infant, a toddler or a young child.

Suitably, the subject of interest is 5 years of age or less, 4 years of age or less, 3 years of age or less, 2 years of age or less, 1 year of age or less, or 0.5 years of age or less. Suitably, the subject of interest is 60 months of age or less, 48 months of age or less, 36 months of age or less, 24 months of age or less, 12 months of age or less, or 6 months of age or less.

Suitably, the subject of interest is 0 years of age or more, 0.5 years of age or more, or 1 year of age or more. Suitably, the subject of interest is 0 months of age or more, 6 months of age or more, or 12 months of age or more.

Suitably, the subject of interest is from 0 years to 5 years of age, from 0 years to 4 years of age, from 0 years to 3 years of age, from 0 years to 2 years of age, or from 0 years to 1 year of age. In some embodiments, the subject of interest is from 0 years to 2 years of age. Suitably, the subject of interest is from 0 months to 60 months of age, from 0 months to 48 months of age, from 0 months to 36 months of age, from 0 months to 24 months of age, or from 0 months to 12 months of age. In some embodiments, the subject of interest is from 0 months to 24 months of age.

Suitably, subject of interest is from 0 years to 1 year of age, 0.5 years to 1 year of age, or 1 year to 2 years of age. Suitably, the subject of interest is from 0 months to 12 months of age, 6 months to 12 months of age, or 12 months to 24 months of age.

Gut Metagenomic Data from a Subject

The subject's gut metagenomic data may be obtained or obtainable by any suitable sampling method described herein. The subject's gut metagenomic data may be obtained or obtainable by the same method as the gut metagenomic data from the population of healthy subjects or by a different method.

The subject's gut metagenomic data may be obtained from or obtainable from a fecal sample, an endoscopy samples (e.g. a biopsy sample, a luminal brush sample, a laser capture microdissection sample), an aspirated intestinal fluid sample, a surgery sample, or by an in vivo model or an intelligent capsule. Preferably, the subject's gut metagenomic data is obtained from a fecal sample.

The gut metagenomic data may be obtained by or obtainable by any suitable method. For example, the gut metagenomic data may be obtained by or obtainable by sequencing methods (e.g. next-generation sequencing (NGS) methods), or any other suitable method. One advantage of the present invention is that next generation sequencing methods may not be required to determine a subject's elaborate gut microbiome status since the few important CAZyme abundances which have the greatest effect on the subject's gut microbiome status can be determined from the trained regression model.

In some embodiments, the subject's gut metagenomic data is obtained by NGS methods.

The gut metagenomic data may provide one or more CAZyme abundances. Suitably, the gut metagenomic data may provide 2 or more (e.g. 2, 3, 4, 5, 6, 7, 8, 9, 10) CAZyme abundances, 3 or more CAZyme abundances, 4 or CAZyme abundances, 5 or more CAZyme abundances, 6 or more CAZyme abundances, 7 or more CAZyme abundances, 8 or more CAZyme abundances, 9 or more CAZyme abundances, 10 or more CAZyme abundances, 20 or more CAZyme abundances, 30 or more CAZyme abundances, 50 or more CAZyme abundances, 50 or more CAZyme abundances, or 100 or more CAZyme abundances. Suitably, the gut metagenomic data may provide 500 or fewer CAZyme abundances, 400 or fewer CAZyme abundances, 200 or fewer CAZyme abundances, 100 or fewer CAZyme abundances, 50 or fewer CAZyme abundances, 40 or fewer CAZyme abundances, 30 or fewer CAZyme abundances, 20 fewer CAZyme abundances, 10 or fewer CAZyme abundances, 9 or fewer CAZyme abundances, 8 or fewer CAZyme abundances, 7 or fewer CAZyme abundances, 6 or fewer CAZyme abundances, 5 or fewer CAZyme abundances, 4 or fewer CAZyme abundances. Suitably, the gut metagenomic data may provide from 2 to 500 CAZyme abundances, from 5 to 200 CAZyme abundances, or from 10 to 100 CAZyme abundances. Suitably, the gut metagenomic data may provide from 2 to 50 CAZyme abundances, or from 5 to 20 CAZyme abundances. The subject's gut metagenomic data may provide the abundance for the same CAZymes as the gut metagenomic data from the population of healthy subjects or for different CAZymes.

### Outlier in a Trained Regression Model

In one aspect, the present invention provides a method for determining the gut microbiome status of a subject, wherein the method comprises determining whether the subject is an outlier or not in a trained regression model. The method may use any trained regression model described herein.

Suitably, the gut microbiome status of the subject is healthy if the subject is not an outlier in the trained regression model, and/or the gut microbiome status of the subject is not healthy if the subject is an outlier in the trained regression model.

In some embodiments, the gut microbiome status of the subject is healthy if the subject is not an outlier in the trained regression model. In this context, a “healthy” gut microbiome status means that the subject has gut metagenome that does not differ significantly from the gut metagenome of a population of healthy subjects.

A “healthy” gut microbiome status may mean that that the subject is in an appropriate gut maturation state, is in an appropriate gut progression state, and/or is in an appropriate gut succession stage.

In one embodiment, a healthy gut microbiome status means that the subject is in an appropriate gut maturation state. An “appropriate gut maturation state” may mean that the subject's gut microbiome is maturing normally or properly.

In one embodiment, a healthy gut microbiome status means that the subject is in an appropriate gut progression state. An “appropriate gut progression state” may mean that the subject's gut microbiome is progressing or evolving in a timely manner.

In one embodiment, a healthy gut microbiome status means that the subject is in an appropriate gut succession state. An “appropriate gut succession state” may mean that the subject's gut microbiome is succeeding in a timely manner.

In some embodiments, the gut microbiome status of the subject is not healthy if the subject is an outlier. In this context, a gut microbiome status which is “not healthy” means that the subject has a gut metagenome that differs significantly from the gut metagenome of a population of healthy subjects.

A gut microbiome status which is “not healthy” may mean that that the subject is not in an appropriate gut maturation state, is not in an appropriate gut progression state, and/or is not in an appropriate gut succession stage. In one embodiment, a gut microbiome status which is not healthy means that the subject is not in an appropriate gut maturation state. In one embodiment, a gut microbiome status which is not healthy means that the subject is not in an appropriate gut progression state. In one embodiment, a gut microbiome status which is not healthy means that the subject is not in an appropriate gut succession state.

Any suitable statistical method may be used to determine whether the subject is an outlier in the trained regression model (see e.g. Hodge, V. and Austin, J., 2004. Artificial intelligence review, 22(2), pp. 85-126). For example, the subject may be determined to be an outlier based on the standard errors, confidence intervals, prediction intervals, and/or standard deviations in the trained regression model. Suitably, the subject may be determined to be an outlier if their gut metagenomic data differs significantly from the trained regression line, based on the standard errors, confidence intervals, prediction intervals, and/or standard deviations of the trained regression line.

Suitable cut-offs will be well known to the skilled person. For example, three standard deviations from the mean is a common cut-off in practice for identifying outliers in a Gaussian or Gaussian-like distribution.

In some embodiments, the subject is determined to be an outlier based on the standard error of the trained regression model. The standard error of the regression (SE) represents the average distance that the observed values fall from the regression line. Suitably, the subject is an outlier if their gut metagenomic data is −2SE or less or 2SE or more, −3SE or less or 3SE or more, or −4SE or less or 4SE or more from the trained regression line. Suitably, the subject is an outlier if their gut metagenomic data is −2SE or less or 2SE or more from the trained regression line.

In some embodiments, the subject is determined to be an outlier based on the confidence interval of the trained regression model. The confidence interval may be determined by any suitable method, for example using resampling approaches (e.g. bootstrap resampling). Suitably, the subject is an outlier if their gut metagenomic data falls outside the 90% confidence interval, the 95% confidence interval, the 98% confidence interval, or the 99% confidence interval in the trained regression model. Suitably, the subject is an outlier if their gut metagenomic data falls outside the 95% confidence interval in the trained regression model.

In some embodiments, the subject is determined to be an outlier based on prediction interval of the trained regression model. Suitably, the subject is an outlier if their gut metagenomic data falls outside the 90% prediction interval, the 95% prediction interval, the 98% prediction interval, or the 99% prediction interval in the trained regression model. Suitably, the subject is an outlier if their gut metagenomic data falls outside the 95% prediction interval in the trained regression model.

In some embodiments, the subject is determined to be an outlier based on standard deviation of the trained regression model. For example, a Z-score can be used to determine whether the subject is an outlier. The Z-score is the number of standard deviations above and below the mean. Suitably, the subject is an outlier if they have a Z-score of −2 or less or 2 or more, a Z-score of −3 or less or 3 or more, or a Z-score of −4 or less or 4 or more in the trained regression model. Suitably, the subject is an outlier if they have a Z-score of −2 or less or 2 or more in the trained regression model.

In some embodiments, the subject is determined to be an outlier if their gut metagenomic data is −2SE or less or 2SE or more from the trained regression line, if their gut metagenomic data falls outside the 95% confidence interval in the trained regression model, if their gut metagenomic data falls outside the 95% prediction interval in the trained regression model, and/or if they have a Z-score of −2 or less or 2 or more in the trained regression model.

In some embodiments, the subject is determined to be an outlier if their gut metagenomic data falls outside the 95% confidence interval in the trained regression model and/or if their gut metagenomic data falls outside the 95% prediction interval in the trained regression model.

In some embodiments, the subject is determined to be an outlier if their gut metagenomic data falls outside the 95% prediction interval in the trained regression model.

### On or Off an ELM Trajectory

In one aspect, the present invention provides a method for determining the gut microbiome status of a subject, wherein the method comprises determining whether the subject is on or off an ELM trajectory. The method may use any trained regression model described herein.

Suitably, the gut microbiome status of the subject is healthy if the subject is on the ELM trajectory, and/or wherein the gut microbiome status of the subject is not healthy if the subject is off the ELM trajectory.

In some embodiments, the gut microbiome status of the subject is healthy if the subject is on the ELM trajectory. In some embodiments, the gut microbiome status of the subject is not healthy if the subject is off the ELM trajectory.

Suitably, the subject is on the ELM trajectory if the subject's gut metagenomic data does not differ significantly from the ELM trajectory and/or wherein the subject is off the ELM trajectory if the subject's gut metagenomic data differs significantly from the ELM trajectory.

Any suitable method may be used to determine whether the subject is on the ELM trajectory. For example, the subject may be determined to be on the ELM trajectory based on the standard errors, confidence intervals, prediction intervals, and/or standard deviations of the ELM trajectory.

In some embodiments, the subject is determined to be off the ELM trajectory based on the standard error (SE) of the ELM trajectory. Suitably, the subject is off the ELM trajectory if their gut metagenomic data is −2SE or less or 2SE or more, −3SE or less or 3SE or more, or −4SE or less or 4SE or more from the ELM trajectory. Suitably, the subject is off the ELM trajectory if their gut metagenomic data is −2SE or less or 2SE or more from the ELM trajectory.

In some embodiments, the subject is determined to be off the ELM trajectory based on the confidence interval of the ELM trajectory. Suitably, the subject is off the ELM trajectory if their gut metagenomic data falls outside the 90% confidence interval, the 95% confidence interval, the 98% confidence interval, or the 99% confidence interval of the ELM trajectory. Suitably, the subject is off the ELM trajectory if their gut metagenomic data falls outside the 95% confidence interval of the ELM trajectory.

In some embodiments, the subject is determined to be off the ELM trajectory based on prediction interval of the ELM trajectory. Suitably, the subject is off the ELM trajectory if their gut metagenomic data falls outside the 90% prediction interval, the 95% prediction interval, the 98% prediction interval, or the 99% prediction interval of the ELM trajectory. Suitably, the subject is off the ELM trajectory if their gut metagenomic data falls outside the 95% prediction interval of the ELM trajectory.

In some embodiments, the subject is determined to be off the ELM trajectory based on standard deviation of the ELM trajectory. For example, a Z-score can be used to determine whether the subject is off the ELM trajectory. Suitably, the subject is an outlier if they have a Z-score of −2 or less or 2 or more, a Z-score of −3 or less or 3 or more, or a Z-score of −4 or less or 4 or more. Suitably, the subject is off the ELM trajectory if they have a Z-score of −2 or less or 2 or more.

In some embodiments, the subject is determined to be off the ELM trajectory if their gut metagenomic data is −2SE or less or 2SE or more from the ELM trajectory, if their gut metagenomic data falls outside the 95% confidence interval of the ELM trajectory, if their gut metagenomic data falls outside the 95% prediction interval of the ELM trajectory, and/or if they have a Z-score of −2 or less or 2 or more.

In some embodiments, the subject is determined to be off the ELM trajectory if their gut metagenomic data falls outside the 95% confidence interval of the ELM trajectory model and/or if their gut metagenomic data falls outside the 95% prediction interval of the ELM trajectory.

In some embodiments, the subject is determined to be off the ELM trajectory if their gut metagenomic data falls outside the 95% prediction interval of the ELM trajectory.

### Predicting Subject's Age

The present invention provides a method for predicting the age of a subject given their gut metagenomic data. The prediction may be based on the assumption that the subject is healthy.

The method may comprise predicting the age of the subject given their gut metagenomic data and a trained regression model. The trained regression model may be any trained regression model described herein and/or maybe obtained or obtainable by any method described herein.

The method may comprise: (a) providing gut metagenomic data from a population of healthy subjects; (b) training a regression model on the gut metagenomic data, wherein the age of the healthy subjects at data collection is regressed on one or more CAZyme abundances provided from the gut metagenomic data; (c) providing gut metagenomic data from a subject of interest; and (d) predicting the age of the subject of interest given their gut metagenomic data and the trained regression model.

The present invention provides a method for determining the gut microbiome status of a subject, wherein the gut microbiome status of the subject is healthy if the predicted age of the subject does not differ significantly from the actual age of the subject and/or wherein the gut microbiome status of the subject is not healthy if the predicted age of the subject differs significantly from the actual age of the subject.

Any suitable method may be used to determine whether the predicted age of the subject differs significantly from the actual age of the subject, for example based on standard errors, confidence intervals, prediction intervals, and/or standard deviations of the trained regression model (as described above in more detail).

Suitably, the predicted age of the subject differs significantly from the actual age of the subject if their predicted age is −2SE or less or 2SE or more, −3SE or less or 3SE or more, or −4SE or less or 4SE or more from their actual age. Suitably, the predicted age of the subject differs significantly from the actual age of the subject if their predicted age is −2SE or less or 2SE or more from their actual age.

Suitably, the predicted age of the subject differs significantly from the actual age of the subject has an age Z-score of −2 or less or 2 or more, an age Z-score of −3 or less or 3 or more, or an age Z-score of −4 or less or 4 or more. Suitably, the predicted age of the subject differs significantly from the actual age of the subject if the subject has an age Z-score of −2 or less or 2 or more.

Suitably, the predicted age of the subject differs significantly from the actual age of the subject if it differs by about 1 month or more (e.g. 1, 2, 3, 4, 5, 6, 7, 8, 9, 10, 11, or 12 months), by about 2 months or more, by about 3 months or more, by about 4 months or more, by about 5 months or more, 6 months or more, by about 7 months or more, by about 8 months or more, by about 9 months or more, by about 10 months or more, by about 11 months or more, or by about 12 months or more. Suitably, the predicted age of the subject differs significantly from the actual age of the subject if it differs by about 0.5 years or more (e.g. 0.5, 0.6, 0.7, 0.8, 0.9, or 1 year), by about 0.6 years or more, by about 0.7 years or more, by about 0.8 years or more, by about 0.9 years or more, or by about 1 year or more.

The present invention also provides use of one or more CAZymes abundances provided from a subject's gut metagenomic data to predict the age of the subject. The present invention also provides use of a trained regression model according to the present invention to predict the age of a subject.

### Method for Maintaining or Improving the Gut Microbiome Status of a Subject

The present invention provides a method for maintaining or improving the gut microbiome status of a subject.

The method may comprise determining the gut microbiome status of the subject using any method described herein and adjusting the diet, nutrient intake, and/or lifestyle of the subject to maintain or improve the subject's gut microbiome status.

After adjusting the diet, nutrient intake, and/or lifestyle of the subject the gut microbiome status of the subject may be healthy. After adjusting the diet, nutrient intake, and/or lifestyle of the subject the subject may be in an appropriate gut maturation state, in an appropriate gut progression state, and/or in an appropriate gut succession stage.

The adjusted diet, nutrient intake, and/or lifestyle of the subject may change one or more CAZyme abundances. Suitably, the CAZyme abundances are ones which have a high impact on the trained regression model and/or ones which differ significantly from the median CAZyme abundances in the trained regression model. In some embodiments, the adjusted diet, nutrient intake, and/or lifestyle of the subject may change CAZyme abundances which have a high impact on the trained regression model and which differ significantly from the median CAZyme abundances in the trained regression model.

Any suitable statistical method may be used to identify CAZyme abundances which have a high impact on the trained regression model, for example based on the feature importance. The feature importance may be determined using any suitable statistical method, for example based on SHapley Additive exPlanation (SHAP) values (Lundberg S M, et al. Nat Mach Intell. 2020).

Any suitable statistical method may be used to identify CAZyme abundances which differ significantly from the median CAZyme abundances in the trained regression model, for example based on standard errors, confidence intervals, prediction intervals, and/or standard deviations (as described above in more detail).

The adjusted diet, nutrient intake, and/or lifestyle of the subject may increase the abundance of favourable CAZymes and/or to decrease the abundance of unfavourable CAZymes.

Suitably, the adjusted diet, nutrient intake, and/or lifestyle of the subject may increase the abundance of one or more favourable CAZyme. In this context, a “favourable CAZymes” may be CAZymes which have a lower abundance in the subject's gut metagenomic data than in a population of healthy subjects. For example, favourable CAZymes may be the CAZymes that have a lower abundance in the subject's gut metagenomic data compared to the median CAZyme abundance in a trained regression model.

Suitably, the adjusted diet, nutrient intake, and/or lifestyle of the subject may decrease the abundance of one or more unfavourable CAZyme. In this context, “unfavourable CAZymes” may be CAZymes which have a higher abundance in the subject's gut metagenomic data than in a population of healthy subjects. For example, unfavourable CAZymes may be the CAZymes that have a higher abundance in the subject's gut metagenomic data compared to the median CAZyme abundance in a trained regression model.

Suitably, after adjusting the diet, nutrient intake, and/or lifestyle of the subject the gut microbiome status of the subject is healthy. The gut microbiome status may be determined using any method described herein (e.g. the same method used to determine the gut microbiome status prior to adjusting the diet, nutrient intake, and/or lifestyle of the subject).

The present invention also provides a method for determining a subject's diet and/or nutrient intake. The method may comprise determining the diet and/or nutrient intake required to maintain or improve the gut microbiome status of the subject.

These personalised recommendations may change over time as the subject's gut microbiome status changes over time. The methods of the present invention may be used one or more times (e.g. 1, 2, 3, 4, 5, 6, 7, 8, 9, 10) or two or more times when the subject of interest is from 0 years to 1 year of age, 0.5 years to 1 year of age, or 1 year to 2 years of age. The methods of the present invention may be used one or more times (e.g. 1, 2, 3, 4, 5, 6, 7, 8, 9, 10) or two or more times when the subject of interest is from 0 months to 12 months of age, 6 months to 12 months of age, or 12 months to 24 months of age. The methods of the present invention may be used two times or more (e.g. 2, 3, 4, 5, 6, 7, 8, 9, 10 times), three times or more, four times or more, or five times or more, in the first 5 years of a subject's life. For example, the methods of the present invention may be used two times or more (e.g. 2, 3, 4, 5, 6, 7, 8, 9, 10 times), three times or more, four times or more, or five times or more, in the first 2 years of life.

### Diet

In one aspect, the present invention provides a method for maintaining or improving the gut microbiome status comprising adjusting the diet of the subject. For example, the subject may be provided a diet recommendation.

In one aspect, the present invention provides a method for determining a subject's diet. The method may comprise determining the diet required to maintain or improve the gut microbiome status of the subject.

Methods of the invention may comprise determining a diet to increase the abundance of one or more favourable CAZymes and/or to decrease the abundance of one or more unfavourable CAZymes.

Methods of the invention may comprise administering a diet to increase the abundance of one or more favourable CAZymes and/or to decrease the abundance of one or more unfavourable CAZymes.

The subject's “diet” may include all the food consumed by the subject. It is known that diet has a major impact on gut microbiome.

The subject's diet may provide a plurality of food groups. The term “food group” may refer to a collection of foods that share similar nutritional properties or biological classifications. Nutrition guides typically divide foods into food groups and Recommended Dietary Allowance recommend daily servings of each group for a healthy diet. Exemplary food groups include fruits; vegetables; pulses, nuts or seeds; meats; starches or grains; dairy; and oils and fats.

The subject's diet may also provide a plurality of food types. The term “food type” may refer to a collection of foods from the same food group that share more similar nutritional properties or biological classifications. Each food group may be further grouped into a plurality of food types. Exemplary food types for the food group fruit can include apples, banana, citrus, berries, other fruits (e.g. pear, peach, pineapple), and dried fruits. Suitable food groups and food types can be readily determined by any suitable method known in the art. For example, suitable food groups and food types can be based on published observations (e.g. Dwyer J T. The Journal of Nutrition. 2018; 148(suppl 3):1575S-80S).

Suitably, the subject's diet may be adjusted by changing the amount of one or more food group and/or one or more food type in the subject's diet. Suitably, the subject is recommended and/or administered food to adjust the diet.

Suitable foods can be identified based on known CAZyme activities and/or CAZyme substrate specificities. For example, to increase the abundance of a favourable CAZyme, the subject's diet can be adjusted to increase the amount of one or more substrate for the CAZyme. CAZyme activities and substrate specificities are available from the CAZy database (http://www.cazy.org) and dbCAN-PUL, which is a database of experimentally characterized CAZyme gene clusters and their substrates (Ausland, C., et al., 2021. Nucleic Acids Research, 49(D1), pp. D523-D528).

The abundance of bacteria in the gut may be related to their ability to break down various carbohydrates, which they do through CAZymes. (El Kaoutari A, et al. Nat Rev Microbiol. 2013). Thus, the abundance of CAZymes can vary based on different factors including food habits (Bhattacharya T, et al. PLoS One. 2015). For infants, diet transition from milk to solid food affects the CAZymes (Ye L, et al. FEMS Microbiol Ecol. 2019). A high-fiber diet increased abundance of 11 microbiome-encoded glycan-degrading CAZymes (Wastyk H C, et al. Cell. 2021). Some other nutritional strategies for CAZymes modulation are discussed in Belzer C, Trends Microbiol. 2022. Experimentally known CAYzmes modulating substrates may fall under categories of beta-glucans & hemicelluloses, alpha-glucans, Algal Glycans, Animal & Fungal Glycans and Other Glycans, the details of which can be found in specialized databases (Ausland C, et al. Nucleic Acids Res. 2021).

In some embodiments, the food comprises one or more CAZyme substrate. In some embodiments, the food comprises one or more carbohydrates. In some embodiments, the food comprises dietary fibre (e.g. carbohydrate polymers, oligomers, and lignin that escape digestion in the small intestine and reach the colon intact). In some embodiments, the food comprises one or more CAZyme-associated carbohydrate and/or one or more CAZyme-associated dietary fibre. As used herein, “CAZyme-associated” carbohydrates and dietary fibre may refer to carbohydrates and dietary fibres which can act as a substrate for one or more CAZymes.

Suitably, the carbohydrates and/or dietary fibre are selected from one or more of: 4-methylumbelliferyl 6-azido-6-deoxy-beta-D-galactoside, N-acetyl-D-galactosamine, N-acetyl-D-glucosamine, N-acetylglucosamine, N-glycan, O-antigen, O-glycan, acarbose, acetylated glucuronoxylan, agar, aldouronate, alginate, alpha-galactoside, alpha-glucan, alpha-mannan, alpha-mannoside, arabinan, arabino-xylooligosaccharide, arabinofuranooligosaccharide, arabinofuranose, arabinogalactan, arabinose, arabinoxylan, beta-galactooligosaccharide, beta-galactoside, beta-glucan, beta-glucoside, beta-mannan, capsule polysaccharide, carboxymethylcellulose, carrageenan, cellobiose, cellodextrin, cellooligosaccharide, cellotriose, cellulose, chitin, chitobiose, chitooligosaccharide, chondroitin disaccharide, chondroitin sulfate, chondrotin sulfate, cyclomaltodextrin, d-galactosamine, dextran, emulsan, exopolysaccharide, fructan, fructooligosaccharide, fucose, fucosyllactose, galactan, galactomannan, galactooligosaccharide, galactose, galacturonic acid, gentiobiose, glucan, glucomannan, glucosamine, glucose, glycogen, glycosaminoglycan, hemicellulose, heparosan, homogalacturonan, host glycan, human milk oligosaccharide, inulin, isomaltose, isomaltotriose, kestose, lacto-n-triose, lactose, laminaribiose, laminarin, levan, lichenan, lignocellulose, lipopolysaccharide, maltodextrin, maltooligosaccharide, maltose, maltotriose, mannan, mannooligosaccharide, mannose, melezitose, melibiose, methylglucuronoarabinoxylan, mucin, oligogalacturonide, outer core capsule polysaccharide, panose, pectin, plant polysaccharide, polygalacturonic acid, polysialic acid, porphyran, pullulan, raffinose-oligosaccharide, raffinose, rhamnogalacturonan, rhamnose, ribose, sialic acid, sialoglycoconjugate, sophorose, sorbitol, stachyose, starch, sucrose, trehalose, ulvan, unsaturated hyaluronate disaccharide, xanthan, xylan, xylobiose, xylodextrin, xyloglucan, xylooligosaccharide, xylose, and xylotriose.

### Nutrient Intake and Supplements

In one aspect, the present invention provides a method for maintaining or improving the gut microbiome status comprising adjusting the nutrient intake of the subject. For example, the subject may be provided a nutrient or supplement recommendation (e.g. meal plans or recipes).

In one aspect, the present invention provides a method for determining a subject's nutrient intake. The method may comprise determining the nutrient intake required to maintain or improve the gut microbiome status of the subject.

Methods of the invention may comprise determining a nutrient intake to increase the abundance of favourable CAZymes and/or to decrease the abundance of unfavourable CAZymes.

Methods of the invention may comprise administering a nutrient or supplement to increase the abundance of favourable CAZymes and/or to decrease the abundance of unfavourable CAZymes.

The term “nutrient” may refer to any substance which is essential for growth and health of a subject. The term nutrient encompasses “macronutrients”, such as carbohydrates, fats and fatty acids, and proteins and “micronutrients”, such as vitamins and minerals. The subject's “nutrient intake” may include all the nutrients consumed by the subject.

Exemplary macronutrients include carbohydrates (including fibre and sugars), protein, and lipids (including long chain polyunsaturated fatty acids). Exemplary micronutrients include vitamins (including vitamin A, vitamin D, vitamin C, folate, vitamin B6, vitamin B12, and vitamin E) and minerals (including sodium, potassium, calcium, iron, zinc, magnesium, and phosphorus).

As used herein, a “supplement” or “dietary supplement” may be used to complement the nutrition of a subject (it is typically used as such but it might also be added to any kind of compositions intended to be ingested by the subject). The supplement may be in any form suitable for intake by the subject and may comprise any suitable nutrients.

Suitably, the subject's nutrient intake may be adjusted by changing the amount of one or more nutrient in the subject's diet and/or by providing a dietary supplement. In some embodiments, the one or more nutrient comprise or consist of one or more carbohydrates. In some embodiments, the one or more nutrient comprise or consist of dietary fibre (e.g. carbohydrate polymers, oligomers, and lignin that escape digestion in the small intestine and reach the colon intact). In some embodiments, the one or more nutrient comprises one or more CAZyme-associated carbohydrate and/or one or more CAZyme-associated dietary fibre.

Suitably, the carbohydrates and/or dietary fibre are selected from one or more of: 4-methylumbelliferyl 6-azido-6-deoxy-beta-D-galactoside, N-acetyl-D-galactosamine, N-acetyl-D-glucosamine, N-acetylglucosamine, N-glycan, O-antigen, O-glycan, acarbose, acetylated glucuronoxylan, agar, aldouronate, alginate, alpha-galactoside, alpha-glucan, alpha-mannan, alpha-mannoside, arabinan, arabino-xylooligosaccharide, arabinofuranooligosaccharide, arabinofuranose, arabinogalactan, arabinose, arabinoxylan, beta-galactooligosaccharide, beta-galactoside, beta-glucan, beta-glucoside, beta-mannan, capsule polysaccharide, carboxymethylcellulose, carrageenan, cellobiose, cellodextrin, cellooligosaccharide, cellotriose, cellulose, chitin, chitobiose, chitooligosaccharide, chondroitin disaccharide, chondroitin sulfate, chondrotin sulfate, cyclomaltodextrin, d-galactosamine, dextran, emulsan, exopolysaccharide, fructan, fructooligosaccharide, fucose, fucosyllactose, galactan, galactomannan, galactooligosaccharide, galactose, galacturonic acid, gentiobiose, glucan, glucomannan, glucosamine, glucose, glycogen, glycosaminoglycan, hemicellulose, heparosan, homogalacturonan, host glycan, human milk oligosaccharide, inulin, isomaltose, isomaltotriose, kestose, lacto-n-triose, lactose, laminaribiose, laminarin, levan, lichenan, lignocellulose, lipopolysaccharide, maltodextrin, maltooligosaccharide, maltose, maltotriose, mannan, mannooligosaccharide, mannose, melezitose, melibiose, methylglucuronoarabinoxylan, mucin, oligogalacturonide, outer core capsule polysaccharide, panose, pectin, plant polysaccharide, polygalacturonic acid, polysialic acid, porphyran, pullulan, raffinose-oligosaccharide, raffinose, rhamnogalacturonan, rhamnose, ribose, sialic acid, sialoglycoconjugate, sophorose, sorbitol, stachyose, starch, sucrose, trehalose, ulvan, unsaturated hyaluronate disaccharide, xanthan, xylan, xylobiose, xylodextrin, xyloglucan, xylooligosaccharide, xylose, and xylotriose.

### Lifestyle

In one aspect, the present invention provides a method for maintaining or improving the gut microbiome status comprising adjusting the lifestyle of the subject. For example, the subject may be provided a lifestyle recommendation.

Methods of the invention may comprise adjusting the subject's lifestyle to increase the abundance of favourable CAZymes and/or to decrease the abundance of unfavourable CAZymes.

By the term “lifestyle” is meant any lifestyle choice made by a subject (or subject's caretakers), and may include dietary intake data, data from questionnaires of lifestyle, motivation or preferences. For example, a lifestyle characteristic may be whether the subject is a vegan or an omnivore, whether the subject is lactose intolerant or not, frequency of physical activity, and/or frequency of sedentary activity. Suitably, the subject's lifestyle may be adjusted by changing the subject's meal frequency and timing and/or frequency of physical activity.

### Abundance of Microbial Taxa

Suitably, the abundance of CAZymes may be adjusted by increasing the abundance and/or function of one or more favourable microbial taxa and/or decreasing the abundance and/or function of one or more unfavourable microbial taxa.

CAZymes will be associated with different microbial taxa (see e.g. the CAZy database), therefore adjusting the level of the microbial taxa can also adjust the abundance of CAZymes. In this context, “favourable microbial taxa” may be microbial taxa which are associated with favourable CAZymes and “unfavourable microbial taxa” may be microbial which are associated with unfavourable CAZymes.

Suitable microbial taxa are known in the art. For example, Bacteroides thetaiotaomicron contains a large number of glycoside hydrolases (GH CAZymes) for glycan utilization such as fructans, mannans and starch (Sonnenburg E D, et al. Proc Natl Acad Sci USA. 2006). Bacteroides contains the most CAZyme families, including 80 GH/PL and 13 CBM families (Ye L, et al. FEMS Microbiol Ecol. 2019). Some of the other bacteria genus that can be modulated to achieve CAZyme profiles are Bifidobacterium, Lactobacillus, Eubacterium, Roseburia, and Ruminococcus (Bhattacharya T, et al. PLoS One. 2015; Flint H J, et al. Gut Microbes. 2012).

Methods of the invention may comprise determining and/or administering a diet to increase the abundance and/or function of one or more favourable microbial taxa and/or to decrease the abundance and/or function of one or more unfavourable microbial taxa. Correlations are known between diet and microbial taxa (see e.g. Wu, G. D., et al., 2011. Science, 334(6052), pp. 105-108). For example, diets with increased fibre may stimulate growth of Bifidobacterium and Lactobacillus (see e.g. Wegh, C. A., et al., 2017. Expert review of gastroenterology & hepatology, 11(11), pp. 1031-1045). Suitably, the subject is recommended and/or administered food to adjust the diet. In some embodiments, the food comprises dietary fibre (e.g. carbohydrate polymers, oligomers, and lignin that escape digestion in the small intestine and reach the colon intact). In some embodiments, the food comprises vitamins, such as Riboflavin (Vitamin B2), Retinol (Vitamin A), and Calciferol (Vitamin D), and/or minerals, such as Manganese, Zinc, and Potassium.

Methods of the invention may comprise determining a nutrient intake and/or administering a nutrient or supplement to increase the abundance and/or function of one or more favourable microbial taxa and/or to decrease the abundance and/or function of one or more unfavourable microbial taxa. Suitably, the subject's nutrient intake may be adjusted by changing the amount of one or more nutrient in the subject's diet and/or by providing a dietary supplement. Correlations are known between nutrients and microbial taxa (see e.g. Wu, G. D., et al., 2011. Science, 334(6052), pp. 105-108). For example, it is known that human milk oligosaccharides can regulate gut microbiota (see e.g. Sela, D. A. and Mills, D. A., 2010. Trends in microbiology, 18(7), pp. 298-307). Suitably, the subject is administered food and/or supplements to adjust the nutrient intake. In some embodiments, the food and/or supplements comprise prebiotics (e.g. human milk oligosaccharides and/or inulin), probiotics, synbiotics, vitamins (e.g. Riboflavin (Vitamin B2), Retinol (Vitamin A), and/or Calciferol (Vitamin D)) and/or minerals (e.g. Manganese, Zinc, and/or Potassium).

In some embodiments, the food and/or supplements comprise prebiotics, probiotics, and/or synbiotics. Any suitable prebiotic, probiotic, and/or synbiotic may be used (see e.g. Thomas, D. W. and Greer, F. R., 2010. Pediatrics, 126(6), pp. 1217-1231).

The term “prebiotic” may refer to a non-digestible component that benefits the subject by selectively stimulating the favourable growth and/or activity of one or more microbial taxa. Exemplary prebiotics include human milk oligosaccharides. Exemplary prebiotic oligosaccharides include galacto-oligosaccharides (GOS), fructo-oligosaccharides (FOS), 2′-fucosyllactose, lacto-N-neo-tetraose, and inulin.

The term “probiotic” may refer to a component that contains a sufficient number of viable microorganisms to alter the gut microbiota of the subject (see e.g. Hill, C., et al., 2014. Nature reviews Gastroenterology & hepatology, 11(8), p. 506). Exemplary probiotic microorganisms may include Escherichia, Bacteroides, Roseburia, Faecalibacterium, Sutterella, SMB53, Collinsella, Ruminococcus, Akkermansia, Veillonella, Parabacteroides, Clostridium, Oscillospira, Megasphaera, Fusobacterium, Citrobacter, Neisseria, Bifidobacterium, Lachnospira, Dialister, Ruminococcus, Blautia, Streptococcus, Eggerthella, Enterococcus and Paraprevotella.

In some embodiments, the probiotic comprises a commercially available probiotic strain and/or a strain which has been shown to have health benefits (See e.g. Fijan, S., 2014. International journal of environmental research and public health, 11(5), pp. 4745-4767). In some embodiments, the probiotic comprises Escherichia, Bifidobacterium, Streptococcus, and/or Enterococcus. In some embodiments, the probiotic comprises one or more strain selected from: E. coli Nissle 1917, B. infantis, B. animalis subsp. lactis, B. bifidum, B. longum, B. breve, S. thermophilus, E. durans, and, E. faecium.

The term “synbiotic” may refer to a component that contains both probiotics and prebiotics (see e.g. Swanson, K. S., et al., 2020. Nature Reviews Gastroenterology & Hepatology, 17(11), pp. 687-701).

In some embodiments, the food and/or supplements comprise vitamins and/or minerals. Dietary guidelines have been established for certain vitamins and minerals.

Exemplary vitamins include vitamin A, vitamin D, vitamin C, folate, vitamin B2, vitamin B6, vitamin B12, and vitamin E. For example, the vitamins may comprise or consist of Riboflavin (Vitamin B2), Retinol (Vitamin A), and/or Calciferol (Vitamin D).

Exemplary minerals include sodium, potassium, calcium, iron, zinc, magnesium, and phosphorus. For example, the minerals may comprise or consist of manganese, zinc, and/or potassium. Minerals are usually used in their salt form.

Methods of the invention may comprise adjusting the subject's lifestyle to increase the abundance and/or function of one or more favourable microbial taxa and/or to decrease the abundance and/or function of one or more unfavourable microbial taxa. Suitably, the subject's lifestyle may be adjusted by changing the subject's meal frequency and timing and/or frequency of physical activity. For example, it is known that a regular meal pattern may modulate gut microbiota (see e.g. Paoli, A., et al., 2019. Nutrients, 11(4), p. 719) and that exercise may exert an influence on gut microbiota (see e.g. O'Sullivan, O., et al., 2015. Gut microbes, 6(2), pp. 131-136).

### Computer Program and Computer-Readable Medium

The methods described may be computer-implemented methods.

In one aspect, the present invention provides a data processing system comprising means for carrying out a method of the invention.

In one aspect, the present invention provides a data processing apparatus comprising a processor configured to perform a method of the invention.

In one aspect, the present invention provides a computer program comprising instructions which, when the program is executed by a computer, cause the computer to carry out a method of the invention.

In one aspect, the present invention provides a computer-readable medium comprising instructions which, when executed by a computer, cause the computer to carry out a method of the invention.

In one aspect, the present invention provides a computer-readable data carrier having stored thereon the computer program of the invention.

In one aspect, the present invention provides a data carrier signal carrying the computer program of the invention.

The systems described herein may display a dashboard or other appropriate user interface to a user that is customized based on the subject of interest. For example, based on the subject's gut metagenomic samples, the subject's determined gut microbiome status, and the subject's personalized advice and recommendations such as nutritional solutions to maintain or improve the subject's gut microbiome status.

### Providing a Trained Regression Model

The methods described herein for determining a trained regression model may be computer-implemented methods.

For example, in one aspect, the present invention provides a computer-implemented method for providing a trained regression model, wherein the method comprises: (a) providing gut metagenomic data from a population of healthy subjects; and (b) training a regression model on the gut metagenomic data, wherein the age of the healthy subjects at data collection is regressed on one or more carbohydrate-active enzyme (CAZyme) abundances provided from the gut metagenomic data.

In one aspect, the present invention provides a data processing system comprising means for determining a trained regression model given gut metagenomic data from a population of healthy subjects providing the age of the healthy subjects at data collection and on one or more CAZymes abundances, as described herein.

In one aspect, the present invention provides a data processing apparatus comprising a processor configured to determine a trained regression model given gut metagenomic data from a population of healthy subjects providing the age of the healthy subjects at data collection and on one or more CAZymes abundances, as described herein.

In one aspect, the present invention provides a computer program comprising instructions which, when the program is executed by a computer, cause the computer to determine a trained regression model given gut metagenomic data from a population of healthy subjects providing the age of the healthy subjects at data collection and on one or more CAZymes abundances, as described herein.

In one aspect, the present invention provides a computer-readable medium comprising instructions which, when executed by a computer, cause the computer to determine a trained regression model given gut metagenomic data from a population of healthy subjects providing the age of the healthy subjects at data collection and on one or more CAZymes abundances, as described herein.

### Predicting the Age of a Subject

The methods described herein predicting the age of a subject may be computer-implemented methods.

For example, in one aspect, the present invention provides a computer-implemented method for predicting the age of a subject, wherein the method comprises: (a) providing a trained regression model according to the present invention; (b) providing gut metagenomic data from the subject; and (c) predicting the age of the subject given their gut metagenomic data and the trained regression model.

In one aspect, the present invention provides a data processing system comprising means for predicting the age of a subject given a trained regression model according to the present invention and their gut metagenomic data, as described herein.

In one aspect, the present invention provides a data processing apparatus comprising a processor configured to predict the age of a subject given a trained regression model according to the present invention and their gut metagenomic data, as described herein.

In one aspect, the present invention provides a computer program comprising instructions which, when the program is executed by a computer, cause the computer to predict the age of a subject given a trained regression model according to the present invention and their gut metagenomic data, as described herein.

In one aspect, the present invention provides a computer-readable medium comprising instructions which, when executed by a computer, cause the computer to predict the age of a subject given a trained regression model according to the present invention and their gut metagenomic data, as described herein.

### Determining the Gut Microbiome Status of a Subject

The methods described herein for determining the gut microbiome status of a subject may be computer-implemented methods.

### Outlier in a Trained Regression Model

In one aspect, the present invention provides a computer-implemented method for determining the gut microbiome status of a subject, wherein the method comprises: (a) providing a trained regression according to the present invention; (b) providing gut metagenomic data from the subject; and (c) determining whether the subject is an outlier or not in the trained regression model; wherein the gut microbiome status of the subject is healthy if the subject is not an outlier in the trained regression model, and/or wherein the gut microbiome status of the subject is not healthy if the subject is an outlier.

In one aspect, the present invention provides a data processing system comprising means for determining the gut microbiome status of a subject given a trained regression according to the present invention and their gut metagenomic data, as described herein.

In one aspect, the present invention provides a data processing apparatus comprising a processor configured to determine the gut microbiome status of a subject given a trained regression according to the present invention and their gut metagenomic data, as described herein.

In one aspect, the present invention provides a computer program comprising instructions which, when the program is executed by a computer, cause the computer to determine the gut microbiome status of a subject given a trained regression according to the present invention and their gut metagenomic data, as described herein.

In one aspect, the present invention provides a computer-readable medium comprising instructions which, when executed by a computer, cause the computer to determine the gut microbiome status of a subject given a trained regression according to the present invention and their gut metagenomic data, as described herein.

### On or Off an ELM Trajectory

In one aspect, the present invention provides a computer-implemented method for determining the gut microbiome status of a subject, wherein the method comprises: (a) providing a trained regression model according to the present invention, wherein the trained regression model is an ELM trajectory; (b) providing gut metagenomic data from the subject; and (c) determining whether the subject is on or off the ELM trajectory; wherein the subject is on the ELM trajectory if the subject's gut metagenomic data does not differ significantly from the ELM trajectory and/or wherein the subject is off the ELM trajectory if the subject's gut metagenomic data differs significantly from the ELM trajectory.

In one aspect, the present invention provides a data processing system comprising means for determining the gut microbiome status of a subject given an ELM trajectory according to the present invention and their gut metagenomic data, as described herein.

In one aspect, the present invention provides a data processing apparatus comprising a processor configured to determine the gut microbiome status of a subject given an ELM trajectory according to the present invention and their gut metagenomic data, as described herein.

In one aspect, the present invention provides a computer program comprising instructions which, when the program is executed by a computer, cause the computer to determine the gut microbiome status of a subject given an ELM trajectory according to the present invention and their gut metagenomic data, as described herein.

In one aspect, the present invention provides a computer-readable medium comprising instructions which, when executed by a computer, cause the computer to determine the gut microbiome status of a subject given an ELM trajectory according to the present invention and their gut metagenomic data, as described herein.

### Predicted Age Differs Significantly

In one aspect, the present invention provides a computer-implemented method for determining the gut microbiome status of a subject, wherein the method comprises predicting the age of the subject by a method according to the present invention, and wherein the gut microbiome status of the subject is healthy if the predicted age of the subject does not differ significantly from the actual age of the subject and/or wherein the gut microbiome status of the subject is not healthy if the predicted age of the subject differs significantly from the actual age of the subject.

In one aspect, the present invention provides a data processing system comprising means for determining the gut microbiome status of a subject given their predicted according to the present invention and their actual age.

In one aspect, the present invention provides a data processing apparatus comprising a processor configured to determine the gut microbiome status of a subject given their predicted according to the present invention and their actual age.

In one aspect, the present invention provides a computer program comprising instructions which, when the program is executed by a computer, cause the computer to determine the gut microbiome status of a subject given their predicted according to the present invention and their actual age.

In one aspect, the present invention provides a computer-readable medium comprising instructions which, when executed by a computer, cause the computer to determine the gut microbiome status of a subject given their predicted according to the present invention and their actual age.

### EXAMPLES

The invention will now be further described by way of examples, which are meant to serve to assist the skilled person in carrying out the invention and are not intended in any way to limit the scope of the invention.

### Example 1—Study Description

Metagenomic sequencing data was obtained for fecal samples from infants of the BCP-enriched study, which is an ancillary study to BCP-study (Howell B R, et al. Neuroimage. 2019) with expanded scope to explore nutritional impacts (https://www.med.unc.edu/neurology/unc-launches-new-study-to-uncover-nutritional-impacts-on-early-brain-development/).

### Example 2—Generating CAZymes Profiles

To determine the relative abundance of CAZymes in each sample a workflow was applied to generate a metagenome assembled genome (MAG) from the metagenomic sequencing data. In more detail, the ATLAS pipeline (Kieser, S., et al., 2020. BMC bioinformatics, 21(1), pp. 1-8) was used where the translated gene products (predicted using Prodigal, Hyatt, D., et al., 2010. BMC bioinformatics, 11(1), pp. 1-11) were clustered to generate non-redundant gene and protein catalogs, which were mapped to the eggNOG catalogue v5 (Huerta-Cepas, J., et al., 2019. Nucleic acids research, 47(D1), pp. D309-D314). For each sample, the relative abundance of CAZymes was determined.

### Example 3—Determining a Reference Set

Similar to selection of reference set of infants in literature (Subramanian S, Huq S, et al. Nature. 2014; Stewart C J, et al. Nature 2018), the inclusion criteria for our Reference set were vaginal delivery mode and term birth (gestational age: 259 to 293 days); and the exclusion criterion was Antibiotics usage. Thus, in this Reference set we had 209 samples from 66 infants (out of total 662 samples from 215 infants). The remaining samples were deemed as non-Reference set.

### Example 4—Derivation of an Early Life Microbiome (ELM) Trajectory

The knowledge and concepts of Early Life Microbiome changes and maturation have been described in Dogra S K, et al. Microorganisms. 2021. The methods to study these Early Life Microbiome changes and maturation have been described in Banjac J, et al. Bioinformatics. 2023. Many other technical details can be found here: https://github.com/JelenaBaniac/microbiome-toolbox We used the Microbiome Toolbox dashboard available at https://microbiome-toolbox.azurewebsites.net/ for the analysis results and figures presented here.

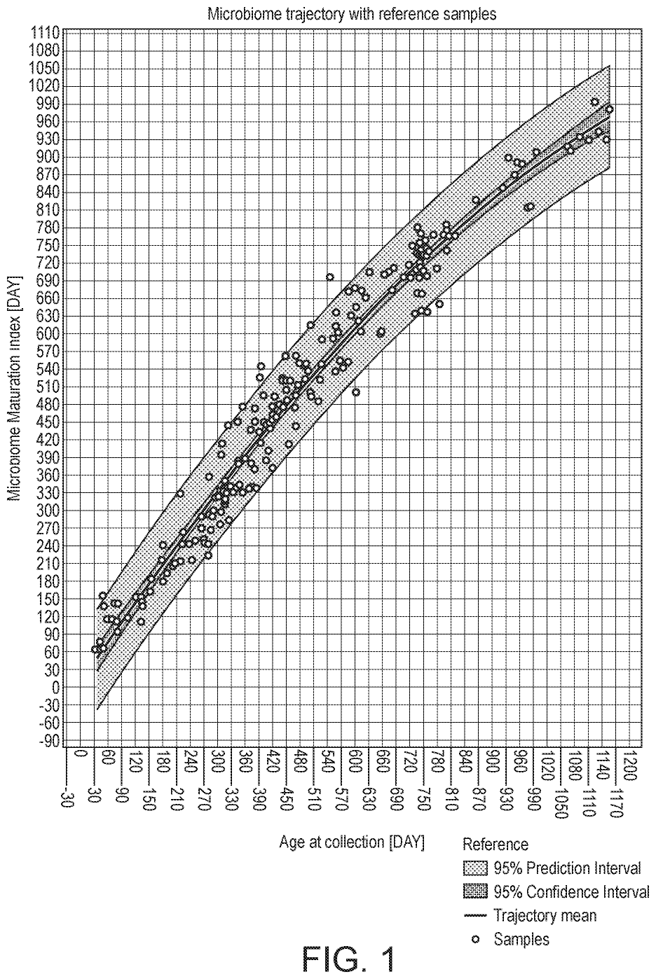

The results obtained for the ELM trajectory are shown in FIG. 1. The model used top 22 features and has a mean absolute error (MAE) of the prediction: 46.362 and percent of variance captured R\hat{ }2: 0.941 These CAZyme features and their relative importance are shown in Table 1.

TABLE 1
CAZyme features constituting the trajectory model

| feature_name | feature_importance |
|---|---|
| GT4 | 0.268989481 |
| GT30 | 0.174987164 |
| GT36 | 0.0695559 |
| GT28 | 0.043591163 |
| GH127 | 0.036952424 |
| GT21 | 0.03585992 |
| GH68 | 0.033368996 |
| GH23 | 0.03323237 |
| GT56 | 0.030868776 |
| CBM50 | 0.027547113 |
| GH3 | 0.024570801 |
| PL11 | 0.024052403 |
| GH9 | 0.023570466 |
| GT35 | 0.023113857 |
| GT17 | 0.022744953 |
| GH102 | 0.021408917 |
| GH32 | 0.020894927 |
| PL13 | 0.0191653 |
| GH26 | 0.018728632 |
| GT10 | 0.017996739 |
| GT51 | 0.014853711 |
| PL12 | 0.013945987 |

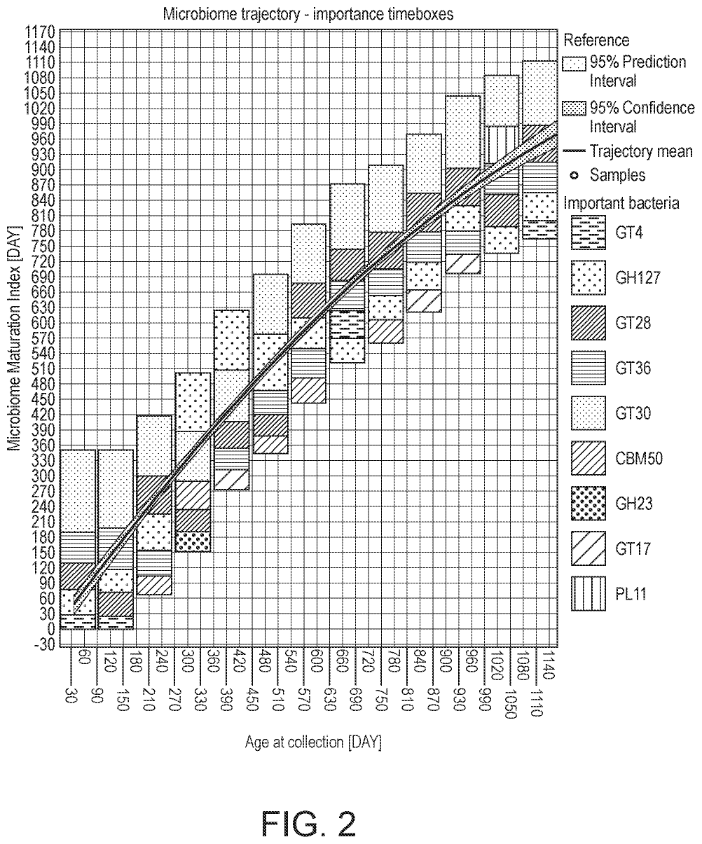

The key CAZyme abundances important in reference trajectory per different time window are shown in FIG. 2. For visualization purposes, the time windows depicted here are of 90 days each to correspond to −3 months. Again, for visualization purposes, only the top 5 important bacteria CAZyme features per time window are shown. Further, for visualization purposes, display of actual samples and the 95% Prediction Interval around them is switched off.

### Example 5—Identification of Samples Off the ELM Trajectory and Correction

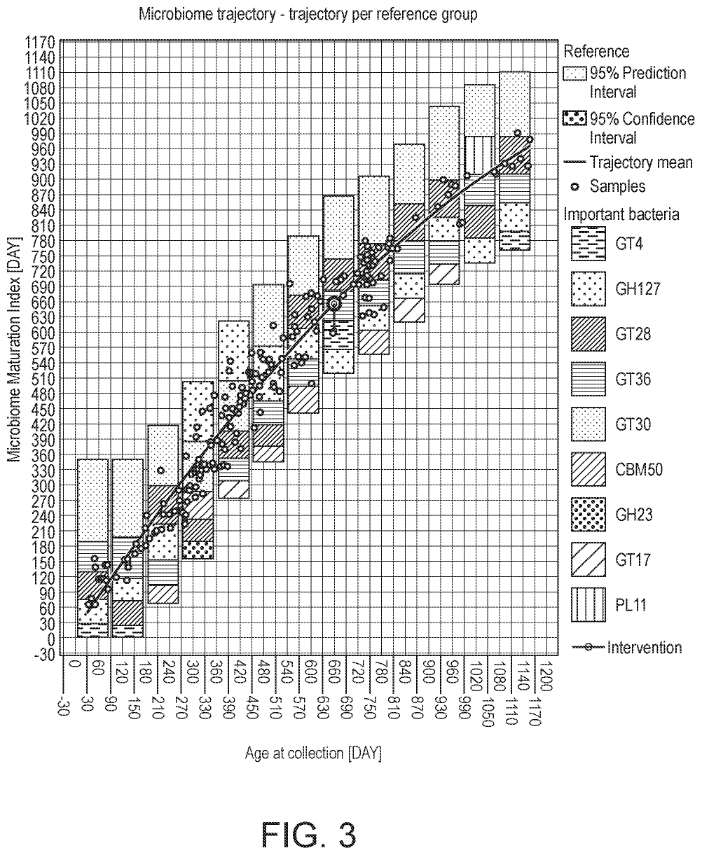

There are some samples (infant at given time-point), who are off the trajectory. An example of off the trajectory sample is shown in FIG. 3. For visualization purposes, the time windows depicted here are of 90 days each to correspond to −3 months. Again, for visualization purposes, only the top 5 important bacteria CAZyme features per time window are shown. Further, for visualization purposes, display of the 95% Confidence Interval and 95% Prediction Interval around the samples is switched off.

The off the ELM trajectory samples do not have key CAZymes in the amounts and ranges found for the healthy reference infants in the same age group. Interventions can be used to restore these CAZymes to their normal amounts and may lead to the sample being placed back on the ELM trajectory.

This is confirmed by simulations where the key CAZyme abundances for the outlier sample were checked against the reference samples in the same age group (time-window). Next, these key CAZyme abundances were changed to be the same as the median for the reference samples in the same age group (time-window). The predictions using the model showed that this can bring the outlier samples back on the ELM trajectory (see FIG. 3) as shown by a drawn line and circle around the center of the figure and labelled as “Intervention” in the legend on the right side.

Restoration of the off the trajectory infants back on to the trajectory can be done by personalized food and dietary advises or nutritional supplements such as vitamins and minerals or administration of prebiotics, probiotics or synbiotics.

### Example 6—Differences in Early Life Microbiome (ELM) Reference Trajectory Vs. Non-Reference Trajectory

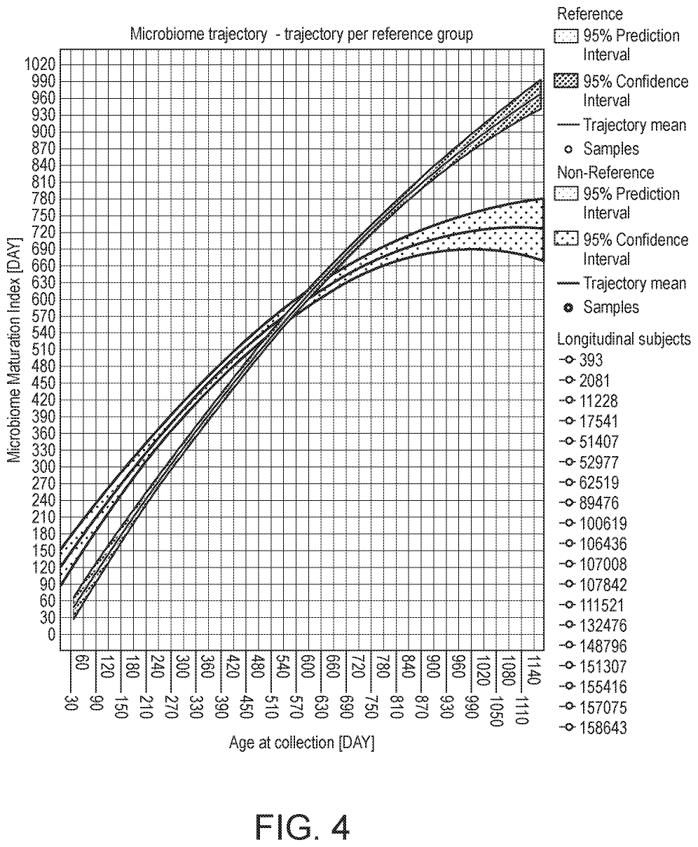

For the trajectory from Reference set of 209 samples (see example 3) the MAE was: 46.362 and R\hat{ }2: 0.941 For the trajectory from non-Reference set of 453 samples the MAE was: 119.442 and R\hat{ }2: 0.599 Statistical p-value for the Reference vs. Non-reference was significant by a spline based test (Shields-Cutler R R, et al. Front Microbiol. 2018): 0.002 Reference trajectory vs. non-Reference trajectory are depicted in FIG. 4. For visualization purposes, display of actual samples and the 95% Prediction Interval around them is switched off.

### Example 7—Statistical Methods

The following formulae were used for defining the fit of the trajectory and determining whether a sample is on or off the ELM trajectory.

### Least Squares Polynomial Fit

$${\{ {\hat{β}}_{j} \}}_{j = 0}^{2} = {arg min}_{\{ {β}_{j} ∈ R \}} E where E = {\sum }_{i = 0}^{n} \vert y ( x \vert {β}_{0} , {β}_{1} , {β}_{2} ) - {y}_{i} \vert$$

n is the number of samples (xᵢ, yᵢ), i∈[0, n] in the dataset.
xᵢ is independent variable (i.e. age at data collection).
yᵢ is dependent variable to be fitted by model function y with 3 parameters and degree 2 (i.e. Microbiome Maturation Index (MMI)).
β₀, β₁, β₂ are parameters of the model to be optimised to minimise E.

### Chi-Square

First, consider yᵢ has uncertainty described by standard deviation σᵢ. Second, consider that residuals r=y(x|β₀,β₁,β₂)−yᵢ˜N. Then the function:

$${\sum }_{i = 1}^{n} {[ \frac{{y}_{i} - y ( x \vert {β}_{1} … {β}_{m} )}{{σ}_{i}} ]}^{2} ∼ {χ}^{2}$$

Once we find the best parameters for optimal fit, the probability distribution of χ² will be χ² distribution for n−2 degrees of freedom. If y1 has no uncertainty, σᵢ=1. This is only used to measure the goodness of a fit. Moreover, we use reduced chi-square χᵣ² where a good fit should have χᵣ²=1.

### Confidence Interval

The 95% probability interval around a fit line contains the mean of new values at a specific age of collection value. Iterative resampling residuals 500 times, where the darker the colour of overlap, the confidence is even higher.

$$\vert {\hat{μ}}_{y \vert x 0} - {μ}_{y \vert x 0} \vert \leq {T}_{n - 2}^{\text{.975}} \hat{σ} \sqrt{\frac{1}{n} + \frac{{( {x}_{0} - \overline{x} )}^{2}}{{{Σ}_{i = 1}^{n} ( {x}_{i} - \overline{x} )}^{2}}}$$

\hat{μ}_{y|x0}=Σᵢ₌₀²aᵢxᵢ is polynomial fit with degree of the polynomial equal to 2.
μ_{y|x0} is the mean response of new values.
x₀ is specific value.
Tₙ₋₂^{0.975} is 97.5^{th} percentile of the Student's t-distribution with n−2 degrees of freedom.
n is number of samples in the dataset.
\hat{σ} is the standard deviation of the error term in the fit:

$$\hat{σ} = {\sum }_{i = 1}^{n} \frac{{( {y}_{i} - \hat{y} )}^{2}}{n - 2}$$

### Prediction Interval

The 95% probability that this shaded interval around fit line contains a new future observation at specific age at collection value.

$$\vert {\hat{y}}_{0} - {y}_{0} \vert \leq {T}_{n - 2}^{\text{.975}} \hat{σ} \sqrt{1 + \frac{1}{n} + \frac{{( {x}_{0} - \overline{x} )}^{2}}{{{Σ}_{i = 1}^{n} ( {x}_{i} - \overline{x} )}^{2}}}$$

ŷ₀=Σᵢ₌₀²aᵢx₀ is polynomial fit with degree of the polynomial equal to 2.
y₀ is the new observation.
x₀ is specific value.
Tₙ₋₂^{0.975} is 97.5^{th} percentile of the Student's t-distribution with n−2 degrees of freedom.
n is number of samples in the dataset.
\hat{σ} is the standard deviation of the error term in the fit:

$$\hat{σ} = \sqrt{{\sum }_{i = 1}^{n} \frac{{( {y}_{i} - \hat{y} )}^{2}}{n - 2}}$$

### Embodiments

Various preferred features and embodiments of the present invention will now be described with reference to the following numbered paragraphs (paras).
1. A method for providing a trained regression model for determining the gut microbiome status of a subject, wherein the method comprises:
   (a) providing gut metagenomic data from a population of healthy subjects; and
   (b) training a regression model on the gut metagenomic data, wherein the age of the healthy subjects at data collection is regressed on one or more carbohydrate-active enzyme (CAZyme) abundances provided from the gut metagenomic data.
2. The method according to para 1, wherein the age of the healthy subjects at data collection is regressed on a plurality of CAZyme abundances provided from the gut metagenomic data.
3. The method according to para 1 or 2, wherein the age of the healthy subjects at data collection is regressed on 5 or more CAZyme abundances, 10 or more CAZyme abundances, 20 or more CAZyme abundances, 50 or more CAZyme abundances, or 100 or more CAZyme abundances.
4. The method according to any preceding para, wherein the method further comprises determining the CAZyme abundances from the gut metagenomic data.
5. The method according to any preceding para, wherein the CAZyme abundances are relative CAZyme abundances, preferably wherein the CAZyme abundances are relative to total bacterial genes.
6. The method according to any preceding para, wherein the CAZymes are microbial CAZymes.
7. The method according to any preceding para, wherein the CAZymes are selected from glycoside hydrolases (GH), glycosyltransferases (GT), Polysaccharide Lyases (PL), Carbohydrate esterases (CE), and their associated carbohydrate binding modules (CBMs).
8. The method according to any preceding para, wherein the CAZymes are classified by clans, families, and/or sub-families, preferably wherein the CAZymes are classified by families.
9. The method according to any preceding para, wherein the CAZymes are selected from one or more of: GH1, GH2, GH3, GH4, GH5, GH6, GH7, GH8, GH9, GH10, GH11, GH12, GH13, GH14, GH15, GH16, GH17, GH18, GH19, GH20, GH21, GH22, GH23, GH24, GH25, GH26, GH27, GH28, GH29, GH30, GH31, GH32, GH33, GH34, GH35, GH36, GH37, GH38, GH39, GH40, GH41, GH42, GH43, GH44, GH45, GH46, GH47, GH48, GH49, GH50, GH51, GH52, GH53, GH54, GH55, GH56, GH57, GH58, GH59, GH60, GH61, GH62, GH63, GH64, GH65, GH66, GH67, GH68, GH69, GH70, GH71, GH72, GH73, GH74, GH75, GH76, GH77, GH78, GH79, GH80, GH81, GH82, GH83, GH84, GH85, GH86, GH87, GH88, GH89, GH90, GH91, GH92, GH93, GH94, GH95, GH96, GH97, GH98, GH99, GH100, GH101, GH102, GH103, GH104, GH105, GH106, GH107, GH108, GH109, GH110, GH111, GH112, GH113, GH114, GH115, GH116, GH117, GH118, GH119, GH120, GH121, GH122, GH123, GH124, GH125, GH126, GH127, GH128, GH129, GH130, GH131, GH132, GH133, GH134, GH135, GH136, GH137, GH138, GH139, GH140, GH141, GH142, GH143, GH144, GH145, GH146, GH147, GH148, GH149, GH150, GH151, GH152, GH153, GH154, GH155, GH156, GH157, GH158, GH159, GH160, GH161, GH162, GH163, GH164, GH165, GH166, GH167, GH168, GH169, GH170, GH171, GH172, GT1, GT2, GT3, GT4, GT5, GT6, GT7, GT8, GT9, GT10, GT11, GT12, GT13, GT14, GT15, GT16, GT17, GT18, GT19, GT20, GT21, GT22, GT23, GT24, GT25, GT26, GT27, GT28, GT29, GT30, GT31, GT32, GT33, GT34, GT35, GT36, GT37, GT38, GT39, GT40, GT41, GT42, GT43, GT44, GT45, GT46, GT47, GT48, GT49, GT50, GT51, GT52, GT53, GT54, GT55, GT56, GT57, GT58, GT59, GT60, GT61, GT62, GT63, GT64, GT65, GT66, GT67, GT68, GT69, GT70, GT71, GT72, GT73, GT74, GT75, GT76, GT77, GT78, GT79, GT80, GT81, GT82, GT83, GT84, GT85, GT86, GT87, GT88, GT89, GT90, GT91, GT92, GT93, GT94, GT95, GT96, GT97, GT98, GT99, GT100, GT101, GT102, GT103, GT104, GT105, GT106, GT107, GT108, GT109, GT110, GT111, GT112, GT113, GT114, PL1, PL2, PL3, PL4, PL5, PL6, PL7, PL8, PL9, PL10, PL11, PL12, PL13, PL14, PL15, PL16, PL17, PL18, PL19, PL20, PL21, PL22, PL23, PL24, PL25, PL26, PL27, PL28, PL29, PL30, PL31, PL32, PL33, PL34, PL35, PL36, PL37, PL38, PL39, PL40, PL41, PL42, CE1, CE2, CE3, CE4, CE5, CE6, CE7, CE8, CE9, CE10, CE11, CE12, CE13, CE14, CE15, CE16, CE17, CE18, CE19, CBM1, CBM2, CBM3, CBM4, CBM5, CBM6, CBM7, CBM8, CBM9, CBM10, CBM11, CBM12, CBM13, CBM14, CBM15, CBM16, CBM17, CBM18, CBM19, CBM20, CBM21, CBM22, CBM23, CBM24, CBM25, CBM26, CBM27, CBM28, CBM29, CBM30, CBM31, CBM32, CBM33, CBM34, CBM35, CBM36, CBM37, CBM38, CBM39, CBM40, CBM41, CBM42, CBM43, CBM44, CBM45, CBM46, CBM47, CBM48, CBM49, CBM50, CBM51, CBM52, CBM53, CBM54, CBM55, CBM56, CBM57, CBM58, CBM59, CBM60, CBM61, CBM62, CBM63, CBM64, CBM65, CBM66, CBM67, CBM68, CBM69, CBM70, CBM71, CBM72, CBM73, CBM74, CBM75, CBM76, CBM77, CBM78, CBM79, CBM80, CBM81, CBM82, CBM83, CBM84, CBM85, CBM86, CBM87, and CBM88.
10. The method according to any preceding para, wherein the trained regression model predicts the age of a subject given their gut metagenomic data.
11. The method according to any preceding para, wherein the trained regression model relates the age of a healthy subject to their microbiome age, microbiome maturation index, and/or microbiome maturation age.
12. The method according to any preceding para, wherein the trained regression model is an Early Life Microbiome (ELM) trajectory.
13. The method according to any preceding para, wherein the trained regression model is for infancy and/or early childhood, preferably wherein the trained regression model is for 0-5 years of age, 0-3 years of age, or 0-2 years of age, more preferably wherein the trained regression model is for 0-24 months of age.
14. The method according to any preceding para, wherein the method further comprises obtaining the gut metagenomic data from the population of healthy subjects.
15. The method according to any preceding para, wherein the gut metagenomic data is obtained from or obtainable from fecal samples and/or by next-generation sequencing.
16. The method according to any preceding para, wherein the gut metagenomic data from a population of healthy subjects comprises at least 50 samples, at least 100 samples, at least 200 samples, at least 300 samples, at least 400 samples, or at least 500 samples.
17. The method according to any preceding para, wherein the healthy subjects are infants and/or children, preferably wherein the healthy subjects are 0-5 years of age, or 0-3 years of age, or 0-2 years of age, more preferably wherein the healthy subjects are 0-24 months of age.
18. The method according to any preceding para, wherein the population of healthy subjects comprises at least 20 healthy subjects, at least 40 healthy subjects, at least 60 healthy subjects, at least 80 healthy subjects, or at least 100 healthy subjects.
19. The method according to any preceding para, wherein the regression model is a tree-based regression model, preferably a random forest regression model.
20. The method according to any preceding para, wherein the age of the healthy subjects at sample collection is also regressed on one or more additional features provided from the gut metagenomic data.
21. A trained regression model obtained or obtainable by a method according to any of paras 1 to 20.
22. A trained regression model for determining the gut microbiome status of a subject given one or more CAZyme abundances provided from the subject's gut metagenomic data.
23. The trained regression model according to para 22, wherein the trained regression model is obtained or is obtainable by a method according to any of paras 1 to 20.
24. A method for predicting the age of a subject, wherein the method comprises:
   (a) providing a trained regression model by a method according to any of paras 1 to 20, or a trained regression model according to any of paras 21 to 23;
   (b) providing gut metagenomic data from the subject; and
   (c) predicting the age of the subject given their gut metagenomic data and the trained regression model.
25. A method for determining the gut microbiome status of a subject, wherein the method comprises:
   (a) providing a trained regression model by a method according to any of paras 1 to 20, or a trained regression model according to any of paras 21 to 23;
   (b) providing gut metagenomic data from the subject; and
   (c) determining whether the subject is an outlier or not in the trained regression model;
wherein the gut microbiome status of the subject is healthy if the subject is not an outlier in the trained regression model, and/or wherein the gut microbiome status of the subject is not healthy if the subject is an outlier in the trained regression model.
26. The method according to para 25, wherein the subject is an outlier based on the standard errors (SE), confidence intervals, prediction intervals, and/or standard deviations in the trained regression model, preferably wherein the subject is an outlier if their gut metagenomic data is −2SE or less or 2SE or more from the trained regression line, if their gut metagenomic data falls outside the 95% confidence interval in the trained regression model, if their gut metagenomic data falls outside the 95% prediction interval in the trained regression model, and/or if they have a Z-score of −2 or less or 2 or more in the trained regression model, more preferably wherein the subject is an outlier if their gut metagenomic data falls outside the 95% prediction interval in the trained regression model.
27. A method for determining the gut microbiome status of a subject, wherein the method comprises:
   (a) providing a trained regression model by a method according to any of paras 1 to 20, or a trained regression model according to any of paras 21 to 23, wherein the trained regression model is an ELM trajectory;
   (b) providing gut metagenomic data from the subject; and
   (c) determining whether the subject is on or off the ELM trajectory;
wherein the gut microbiome status of the subject is healthy if the subject is on the ELM trajectory, and/or wherein the gut microbiome status of the subject is not healthy if the subject is off the ELM trajectory.
28. The method according to para 27, wherein the subject is on the ELM trajectory if the subject's gut metagenomic data does not differ significantly from the ELM trajectory and/or wherein the subject is off the ELM trajectory if the subject's gut metagenomic data differs significantly from the ELM trajectory.
29. The method according to para 27 or 28, wherein the subject is determined to be off the ELM trajectory based on the standard errors (SE), confidence intervals, prediction intervals, and/or standard deviations of the ELM trajectory, preferably the subject is determined to be off the ELM trajectory if their gut metagenomic data is −2SE or less or 2SE or more from the ELM trajectory, if their gut metagenomic data falls outside the 95% confidence interval of the ELM trajectory, if their gut metagenomic data falls outside the 95% prediction interval of the ELM trajectory, and/or if they have a Z-score of −2 or less or 2 or more, more preferably wherein the subject is determined to be off the ELM trajectory if their gut metagenomic data falls outside the 95% prediction interval of the ELM trajectory.
30. A method for determining the gut microbiome status of a subject, wherein the method comprises predicting the age of the subject by a method according to para 24, and wherein the gut microbiome status of the subject is healthy if the predicted age of the subject does not differ significantly from the actual age of the subject and/or wherein the gut microbiome status of the subject is not healthy if the predicted age of the subject differs significantly from the actual age of the subject.
31. The method according to para 30, wherein the predicted age of the subject differs significantly from the actual age of the subject if the predicted age of the subject differs significantly from the actual age of the subject if it differs by about 0.1 years or more, by about 0.2 years or more, by about 0.3 years or more, by about 0.4 years or more, by about 0.5 years or more, by about 0.6 years or more, by about 0.7 years or more, by about 0.8 years or more, by about 0.9 years or more, or by about 1 year or more.
32. The method according to any of paras 24 to 31, wherein the subject's gut metagenomic data is obtained from or obtainable from fecal samples.
33. The method according to any of paras 24 to 32, wherein the subject's gut metagenomic data is obtained or obtainable by next-generation sequencing.
34. The method according to any of paras 24 to 33, wherein the subject's gut metagenomic provides 5 or more CAZyme abundances, 10 or more CAZyme abundances, 20 or more CAZyme abundances, 50 or more CAZyme abundances, or 100 or more CAZyme abundances.
35. A method for maintaining or improving the gut microbiome status of a subject, wherein the method comprises:
   (a) determining the gut microbiome status of the subject by a method according to any of paras 25 to 34; and
   (b) adjusting the diet, nutrient intake, and/or lifestyle of the subject to maintain or improve the subject's gut microbiome status.
36. The method according to para 35, wherein after adjusting the diet, nutrient intake, and/or lifestyle of the subject the gut microbiome status of the subject is healthy.
37. The method according to para 35 or 36, wherein the adjusted diet, nutrient intake, and/or lifestyle of the subject increases the abundance of favourable CAZymes and/or decreases the abundance of unfavourable CAZymes.
38. A method for determining a subject's diet and/or nutrient intake, wherein the method comprises:
   (a) determining the gut microbiome status of the subject by a method according to any of paras 25 to 34; and
   (b) determining the diet and/or nutrient intake required to maintain or improve the gut microbiome status of the subject.
39. The method according to any of paras 35 to 38, wherein the subject is administered food and/or supplements to increase the abundance of favourable CAZymes and/or to decrease the abundance of unfavourable CAZymes.
40. The method according to para 39, wherein the food and/or supplements comprise one or more carbohydrates.
41. The method according to para 40, wherein the carbohydrates are selected from one or more of: 4-methylumbelliferyl 6-azido-6-deoxy-beta-D-galactoside, N-acetyl-D-galactosamine, N-acetyl-D-glucosamine, N-acetylglucosamine, N-glycan, O-antigen, O-glycan, acarbose, acetylated glucuronoxylan, agar, aldouronate, alginate, alpha-galactoside, alpha-glucan, alpha-mannan, alpha-mannoside, arabinan, arabino-xylooligosaccharide, arabinofuranooligosaccharide, arabinofuranose, arabinogalactan, arabinose, arabinoxylan, beta-galactooligosaccharide, beta-galactoside, beta-glucan, beta-glucoside, beta-mannan, capsule polysaccharide, carboxymethylcellulose, carrageenan, cellobiose, cellodextrin, cellooligosaccharide, cellotriose, cellulose, chitin, chitobiose, chitooligosaccharide, chondroitin disaccharide, chondroitin sulfate, chondrotin sulfate, cyclomaltodextrin, d-galactosamine, dextran, emulsan, exopolysaccharide, fructan, fructooligosaccharide, fucose, fucosyllactose, galactan, galactomannan, galactooligosaccharide, galactose, galacturonic acid, gentiobiose, glucan, glucomannan, glucosamine, glucose, glycogen, glycosaminoglycan, hemicellulose, heparosan, homogalacturonan, host glycan, human milk oligosaccharide, inulin, isomaltose, isomaltotriose, kestose, lacto-n-triose, lactose, laminaribiose, laminarin, levan, lichenan, lignocellulose, lipopolysaccharide, maltodextrin, maltooligosaccharide, maltose, maltotriose, mannan, mannooligosaccharide, mannose, melezitose, melibiose, methylglucuronoarabinoxylan, mucin, oligogalacturonide, outer core capsule polysaccharide, panose, pectin, plant polysaccharide, polygalacturonic acid, polysialic acid, porphyran, pullulan, raffinose-oligosaccharide, raffinose, rhamnogalacturonan, rhamnose, ribose, sialic acid, sialoglycoconjugate, sophorose, sorbitol, stachyose, starch, sucrose, trehalose, ulvan, unsaturated hyaluronate disaccharide, xanthan, xylan, xylobiose, xylodextrin, xyloglucan, xylooligosaccharide, xylose, and xylotriose.
42. A data processing system comprising means for carrying out the method according to any of paras 1 to 20 or 25 to 38.
43. A data processing system comprising a processor configured to perform the method according to any of paras 1 to 20 or 25 to 38.
44. A computer program comprising instructions which, when the program is executed by a computer, cause the computer to carry out the method according to any of paras 1 to 20 or 25 to 38.
45. A computer program comprising instructions which, when the program is executed by a computer, cause the computer to determine a trained regression model for determining the gut microbiome status of a subject from a population of healthy subjects, given the age of the healthy subjects at data collection and one or more CAZyme abundances provided from the healthy subject's gut metagenomic data.
46. A computer program comprising instructions which, when the program is executed by a computer, cause the computer to predict the age of a subject or determine the gut microbiome status of a subject, given one or more CAZyme abundances provided from the subject's gut metagenomic data.
47. A computer program comprising instructions which, when the program is executed by a computer, cause the computer to predict the age of a subject or determine the gut microbiome status of a subject, given a regression model trained on the gut metagenomic data from a population of healthy subjects and the subject's gut metagenomic data, wherein the regression model was trained by regressing the age of the healthy subjects at data collection on one or more CAZyme abundances provided from the healthy subject's gut metagenomic data.
48. The computer program according to para 47, wherein the trained regression model is a trained regression model according to any of paras 21 to 23.
49. A computer-readable medium comprising instructions which, when executed by a computer, cause the computer to carry out the method according to any of paras 1 to 20 or 25 to 38.
50. A computer-readable medium comprising instructions which, when executed by a computer, cause the computer to determine a trained regression model for determining the gut microbiome status of a subject from a population of healthy subjects, given the age of the healthy subjects at data collection and one or more CAZyme abundances provided from the healthy subject's gut metagenomic data.
51. A computer-readable medium comprising instructions which, when the program is executed by a computer, cause the computer to predict the age of a subject or determine the gut microbiome status of a subject, given one or more CAZyme abundances provided from the subject's gut metagenomic data.
52. A computer-readable medium comprising instructions which, when executed by a computer, cause the computer to predict the age of a subject or determine the gut microbiome status of a subject, given a regression model trained on the gut metagenomic data from a population of healthy subjects and the subject's gut metagenomic data, wherein the regression model was trained by regressing the age of the healthy subjects at data collection on one or more CAZyme abundances provided from the healthy subject's gut metagenomic data.
53. The computer-readable medium according to para 52, wherein the trained regression model is a trained regression model according to any of paras 21 to 23.
54. A computer-readable data carrier having stored thereon a computer program according to any of paras 44 to 48.
55. A data carrier signal carrying a computer program according to any of paras 44 to 48.
56. Use of one or more CAZyme abundances provided from a subject's gut metagenomic data to predict the age of the subject or to determine the gut microbiome status of the subject.
57. Use of one or more CAZyme abundances provided from gut metagenomic data from a population of healthy subjects to train a regression model.
58. Use of a trained regression model according to any of paras 21 to 23 to predict the age of a subject or to determine the gut microbiome status of a subject.
59. The method according to any of paras 35-41, the data processing system according to para 42 or 43, the computer program according to any of paras 44-48, the computer-readable medium according to any of paras 49-53, the computer-readable data carrier according to para 54, the data carrier signal according to para 55, or the use according to any of paras 56-58, wherein the subject is an infant or a child, preferably wherein the subject is 0-5 years of age, or 0-3 years of age, or 0-2 years of age, more preferably wherein the subject is 0-24 months of age.

All publications mentioned in the above specification are herein incorporated by reference. Various modifications and variations of the disclosed methods, compositions and uses of the invention will be apparent to the skilled person without departing from the scope and spirit of the invention. Although the invention has been disclosed in connection with specific preferred embodiments, it should be understood that the invention as claimed should not be unduly limited to such specific embodiments. Indeed, various modifications of the disclosed modes for carrying out the invention, which are obvious to the skilled person are intended to be within the scope of the following claims.

1. A method for providing a trained regression model for determining the gut microbiome status of a subject, wherein the method comprises:
(a) providing gut metagenomic data from a population of healthy subjects; and
(b) training a regression model on the gut metagenomic data, wherein the age of the healthy subjects at data collection is regressed on one or more carbohydrate-active enzyme (CAZyme) abundances provided from the gut metagenomic data.

2. The method according to claim 1, wherein the CAZymes are selected from the group consisting of glycoside hydrolases (GH), glycosyltransferases (GT), Polysaccharide Lyases (PL), Carbohydrate esterases (CE), and their associated carbohydrate binding modules (CBMs).

3. The method according to claim 1, wherein the method further comprises obtaining the gut metagenomic data from the population of healthy subjects.

4. The method according to claim 1, wherein the healthy subjects are infants and/or children.

5. The method according to claim 1, wherein the regression model is a tree-based regression model, preferably a random forest regression model.

6-7. (canceled)

8. A method for determining the gut microbiome status of a subject, wherein the method comprises:
(a) providing a trained regression model by a method comprising:
providing gut metagenomic data from a population of healthy subjects; and
training a regression model on the gut metagenomic data, wherein the age of the healthy subjects at data collection is regressed on one or more carbohydrate-active enzyme (CAZyme) abundances provided from the gut metagenomic data;
(b) providing gut metagenomic data from the subject; and
(c) determining whether the subject is an outlier or not in the trained regression model;
wherein the gut microbiome status of the subject is healthy if the subject is not an outlier in the trained regression model, and/or wherein the gut microbiome status of the subject is not healthy if the subject is an outlier in the trained regression model.

9. A method for maintaining or improving the gut microbiome status of a subject, wherein the method comprises:
(a) determining the gut microbiome status of the subject by a method comprising:
providing a trained regression model by a method comprising:
providing gut metagenomic data from a population of healthy subjects; and
training a regression model on the gut metagenomic data, wherein the age of the healthy subjects at data collection is regressed on one or more carbohydrate-active enzyme (CAZyme) abundances provided from the gut metagenomic data:
providing gut metagenomic data from the subject; and
determining whether the subject is an outlier or not in the trained regression model;
wherein the gut microbiome status of the subject is healthy if the subject is not an outlier in the trained regression model, and/or wherein the gut microbiome status of the subject is not healthy if the subject is an outlier in the trained regression model; and
(b) adjusting the diet, nutrient intake, and/or lifestyle of the subject to maintain or improve the subject's gut microbiome status.

10. The method according to claim 9, wherein the subject is administered food and/or supplements to increase the abundance of favourable CAZymes and/or to decrease the abundance of unfavourable CAZymes.

11-15. (canceled)
